(19) United States
(12) Patent Application Publication
(10) Pub. No.: US 2023/0226100 A1
(43) Pub. Date: Jul. 20, 2023

(54) CYCLODEXTRIN BASED ANTI-MICROBIAL THERAPY

(71) Applicant: University of Southern California, Los Angeles, CA (US)

(72) Inventors: Stan G. LOUIE, Los Angeles, CA (US); Mark S. HUMAYUN, Los Angeles, CA (US); Isaac ASANTE, Los Angeles, CA (US); Gianluca LAZZI, Los Angeles, CA (US); Jean-Marie Charles Bouteiller, Los Angeles, CA (US)

(21) Appl. No.: 17/996,452

(22) PCT Filed: May 3, 2021
(86) PCT No.: PCT/US2021/030447

§ 371 Date: Oct. 18, 2022

Related U.S. Application Data
(60) Provisional application No. 63/018,920, filed on May 1, 2020.

(51) Int. Cl. A61K 31/724; A61K 47/38; A61P 31/14; A61K 9/00; A61K 9/16; A61K 9/06

(52) U.S. Cl. CPC A61K 31/724; A61K 47/38; A61P 31/14; A61K 9/006; A61K 9/0043; A61K 9/0048; A61K 9/1617; A61K 9/06

(57) ABSTRACT

The disclosure provides a rapidly deployable nanoscale biodegradable system using hydroxypropyl beta cyclodextrin based combination product. Cyclodextrin is an amphiphilic polymer suitable to develop an agnostic barrier blocking pathogenic mi-crobes that has localized on the mucocutaneous lining of the conjunctiva, mouth and nose, lung, or gastrointestinal tract. The cyclodex-trin may bind the viral particles and/or disrupt viral entry mechanisms by removing cholesterol from viral particles to reduce infectivity. Cyclodextrins also may facilitate removal of the viral cholesterol molecules, thus rendering them less viable. Cyclodextrin activity may be further enhanced when used in combination with certain minerals and/or antioxidant compounds.

### RELATED APPLICATIONS

This application claims priority under 35 U.S.C. § 119(e) to U.S. Provisional Patent Application No. 63/018,920, filed May 1, 2020, which is incorporated herein by reference.

### BACKGROUND

Infectious disease outbreaks, particularly those of viral nature, have increased in recent decades, which includes the 2002 SARS-CoV-1 outbreak, 2009 H1N1 flu outbreak, and 2012 MERS-CoV outbreak. Most recently, the Coronavirus disease 2019 (COVID-19) caused by the novel SARS-CoV-2 virus has received widespread attention as the viral pathogen causing this pandemic. In particular, the SARS-CoV2 is highly virulent and transmissible. Moreover, it has been reported that a large percentage of COVID-19 positive patients exhibit mild to undetectable symptoms, thus masking the clinical symptoms.

The epithelial lining of the human nasopharynx serves as a major gateway for viral entry and subsequent infection. Viral infection of host cells is mediated through the interaction of SARS-CoV-2 spike (S) protein receptor binding domain (RBD) with angiotensin converting enzyme-2 (ACE2) found on the surface of a host cell. In situ RNA mapping has revealed ACE2 expression to be highest in the nasal cavity and the respiratory system. This affirms current data pointing to the nasal cavity as a major route for infection leading to pulmonary involvement. Furthermore, scRNA-seq datasets reveal ACE2 and its associated protease transmembrane protease serine 2 (TMPRSS2) are highly expressed in the ciliated and goblet cells found in the nasal cavity.

Transmission of SARS-CoV-2 occurs through direct contact with an infectious individual that generate respiratory droplets or aerosols into the environment. Current preventative strategies for COVID-19 include social distancing, handwashing, and the use of face masks. However, compliance with these guidelines continues to be a challenge for some.

Accordingly, there is a need to reduce this public health burden by lowering the risk of infection and permit the workforce to return to work without the fear of potential infection from this deadly virus. One of the best defenses against the deadly virus and other microbes will be a protective barrier for the mucocutaneous membranes, which consists of one or more layers of epithelial cells that line many tracts and structures of the body, including the mouth, nose, eyelids, trachea (windpipe) and lungs. More specifically, there is an urgent need for a nanoscale biocompatible coating capable of enhancing the mucocutaneous lining found in conjunctival, nasal, oropharyngeal, and gastrointestinal systems having the potential to be effective in preventing microbial attachment onto epithelial cells, thus blocking microbial attachment, entry, and infection. The present invention satisfies these needs.

### SUMMARY

The ability to develop a biodegradable nasopharyngeal barrier can be an effective therapeutic strategy to reduce viral transmission at the point of entry as well as any vaporized or aerosolized chemical agent exposure. Accordingly, the disclosure provides for a rapidly deployable, nanoscale biodegradable system using cyclodextrin-based formulations (e.g., hydroxypropyl beta cyclodextrins (HPBCD)) to form a protective coating or barrier over the surface of cells or mucocutaneous membranes to reduce antimicrobial infection and/or chemical mediated injuries. HPBCD is an FDA-approved inert material (excipients), well suited for oral, intravenous, subcutaneous, nasal spray, or inhaled administration of up to 20% (w/v).

Cyclodextrins comprise a family of amphiphilic polymers, thus providing a suitable platform to develop an agnostic barrier blocking pathogenic microbes localized on the mucocutaneous lining such as found in the conjunctiva, oropharyngeal, nasopharyngeal, bronchial, and gastrointestinal systems. The hydrophobic pockets found in these cyclodextrins not only prevent viral particle binding onto epithelial cells but can also disrupt viral entry mechanisms by removing cholesterol from viral particles thereby reducing infectivity. Further, HPBCD or other disclosed cyclodextrins can act as a nanoscale barrier to prevent viruses—and microbes in general—to attach to host cells such as, but not limited to, the human conjunctival, oropharyngeal, nasopharyngeal, bronchial, and gastrointestinal epithelial cells.

Accordingly, embodiments of the invention provide a particulate nano-formulation for reducing the risk of a microbial infection and chemical-induced injuries comprising a cyclodextrin and a pharmaceutically acceptable carrier.

In certain embodiments of the invention, the cyclodextrin is one or more of hydroxypropyl beta cyclodextrins (HPBCD) i.e., (2-Hydroxypropyl)-Beta-Cyclodextrin), 2-Hydroxypropyl-Gamma-cyclodextrin (HPGCD), crystalline methylated beta-cyclodextrin (CRYSMEB), and sulfobutyl ether-beta-cyclodextrin (SBEBCD).

In certain embodiments, the formulation is 20% or less of the total weight of the formulation, and more preferably, from about 2.5% to about 20% by weight.

In some embodiments of the invention, the cyclodextrin formulation may include a thickening agent. In some embodiments, the ratio of the cyclodextrin to the thickening agent is about 5:3 to about 15:1 by weight. Preferably, the thickening agent, such as, but not limited to hydroxyethyl cellulose (HEC) at the disclosed concentration and proportions can further improve effectiveness and/or safety of the product.

Preferably, embodiments of the invention comprise 0.9% (w/v of the formulation) or less of zinc or zinc containing compound.

Embodiments of the invention also provide for an intranasal, oropharyngeal, oral, rectal, inhaled, intranasal, buccal/sublingual, tablets, capsules, pessaries, eyedrop, or eye wash delivery of the biocompatible cyclodextrin-based formulation to form a barrier or coating on, or in conjunction with, the mucocutaneous lining of a susceptible cell and prevent a viral, fungal, and/or bacterial infection of the susceptible cells.

Preferably, the formulation contacts the epithelial cells of the mucocutaneous lining of a surface of one or more of a nasal, an oropharyngeal, a pulmonary, a bronchial, a gastrointestinal, a rectal, a vaginal, or an ocular cavity.

Certain embodiments of the invention are effective in preventing microbial infections such as bacterial, fungal, parasite, and, in particular, viral infections. Exemplary viral agents include but are not limited to human immunodeficiency virus (HIV), human metapneumovirus (HMPV), parainfluenza virus type 3 (HPIV3), Influenza virus A, Influenza virus B, Influenza virus C, Influenza virus D, coronavirus infectious bronchitis virus (IBV), herpes simplex virus 1 (HSV-1), herpes simplex II (HSV-2) varicella-zoster virus (VZV), cytomegalovirus (CMV), Epstein Bane virus (EBV). Kaposi's Sarcoma herpes virus (KSHS), hepatitis A virus (HAV), hepatitis C virus (HCV), hepatitis B virus (HBV), human papilloma virus (HPV), SARS-CoV-1, Middle East Respiratory Syndrome (MERS) virus, and SARS-CoV-2 virus. Preferably, the invention is effective against SARS-CoV-2 virus and its variants.

In certain embodiments of the invention, the formulation is an aerosol, spray, capsule, tablet, sublingual/buccal tablet or film, inhalant, eyedrop, cream, ointment, wash or foam that may be used to form a protective barrier against microbial infection(s) when the formulation contacts the epithelial cells (or the mucocutaneous lining) of the nasal passage, oropharyngeal passage, bronchial-pulmonary passage, gastrointestinal passage, vaginal-cervical passage or an ocular surface such as the conjunctiva. In other embodiments, the formulation may be in the form of a drop or ointment such as for the use for the conjunctiva of the eye.

Embodiments of the invention also provide a method of reducing a risk of microbial infection comprising contacting cells susceptible to infection with an effective amount of a formulation comprising a cyclodextrins or a cyclodextrin derivative and a pharmaceutically acceptable carrier and excipients such that the application of the formulation to the cells forms a protective coating and/or barrier that prevents the microbe from contacting and entering the cells and/or remove cholesterol from the membrane of the microbial agent to reduce the risk of microbial infections.

In some embodiments of the invention, the formulation may be delivered to the cells using a specialized device that guides the coverage of the susceptible areas such as, but not limited to, an aerosol dispenser or a delayed release apparatus.

These and other features and advantages of this invention will be more fully understood from the following detailed description of the invention taken together with the accompanying claims. It is noted that the scope of the claims is defined by the recitations therein and not by the specific discussion of features and advantages set forth in the present description.

### BRIEF DESCRIPTION OF THE DRAWINGS

The following drawings form part of the specification and are included to further demonstrate certain embodiments or various aspects of the invention. In some instances, embodiments of the invention can be best understood by referring to the accompanying drawings in combination with the detailed description presented herein. The description and accompanying drawings may highlight a certain specific example, or a certain aspect of the invention. However, one skilled in the art will understand that portions of the example or aspect may be used in combination with other examples or aspects of the invention.

FIG. 1. The pH of the various cyclodextrin (CD) derivatives in distilled water. Their pH ranges of 4.5 to 6.8.

FIG. 2. (A). Deposition of particles in the nasal cavity. Particles of different sizes tend to have different preferred deposition regions. (B). Quantification of the rostrocaudal deposition profile for all particles. (C). Quantification of the rostrocaudal deposition profile for particles of different sizes. These results indicate that as the diameter of the particles increases, the proportion of particles deposited in the anterior portion of the nasal cavity increases. Smaller particles tend to be deposited on the lower portion of the nasopharynx.

FIG. 3. Example of results obtained while determining the optimal combination of parameter values to maximize deposition. The three subplots show the percentage of the surface nasal cavity protected as a function of spray head insertion depth (Y axis, in mm) and spray velocity (X axis, in m/s) for three different spray cone angles (π/2, π/3, π/4). For these results, mean spray particle diameter is 50 μm on a lognormal particle distribution function; number of particles is 300,000; sprayed volume is 80 μl. For these parameters, optimal profile was found with a spray velocity of 18m/s, insertion depth of 10 mm and a spray cone angle of π/3.

FIG. 4. Example of results obtained with an inhalation speed of 2 m/s. The four subplots show the percentage of the surface nasal cavity protected as a function of spray head insertion depth (Y axis, in mm) and spray velocity (X axis, in m/s) for four different spray cone angles (π/2, π/3, π/4 and π/6). The mean spray particle diameter is 50 μm on a lognormal particle distribution function; number of particles is 300,000; sprayed volume is 80 μl. For these parameters, an optimal profile resulting in 21% deposition is obtained with a spray velocity of 4 m/s, insertion depth of 10 mm and a spray cone angle of π/2.

FIG. 5. Chemical dye penetration in the presence of HPBCD. An assessment of HPBCD as a barrier in preventing chemical dye penetration of HEK293T cells. The plot indicated the efficacy of the HPBCD nano formulation barrier at reducing the penetration of the dye to the cells (measured by relative fluorescence intensity) compared to media control. All treatments using nanoscale barriers were able to prevent Alamar blue penetration of epithelial cells, where the difference was statistically significant reduction (P<0.01) as compared to no treatment.

FIG. 6. Barrier efficacy in reducing lentivirus infection after 6 hours of incubation. HEK293T cells infected with pLV[Exp]-Puro-CMV>EGFP over 6 hours in the presence 0%-20% HPBCD (w/v) at MOI of 5. Bars indicate the percent relative fluorescence in viral infectivity in the presence of HPBCD only.

FIG. 7. Barrier efficacy in reducing lentivirus infectivity. HEK293T were infected with pLV[Exp]-Puro-CMV>EGFP at MOI of 5 for 0.or 2 hours in the presence 0%-10% HPBCD (w/v). Bars indicate the percent relative fluorescence in viral infectivity in the presence of HPBCD only.

FIG. 8. In vitro viral infectivity study: In vitro viral infectivity study: Fluorescent microscopy of Enhanced Green Fluorescent Protein (EGFP)-expressing lentiviral (MOI=5). Decrease in fluorescence from lentiviral infection was seen with increasing concentration of HPBCD.

FIG. 9. The pH of the various cyclodextrin (CD) barriers with and without zinc and ascorbic acid additives. Viral infectivity and formulation mucocutaneous safety is expected to be optimal between the pH range of 4.5 to 6.5. Each CD was concentrations at 10% w/v and the Zn was present at a concentration of 3 mg/mL.

FIG. 10. HPBCD Barrier is less effective when formulations are admixed with zinc and ascorbic acid additives than without the additives. Viral infectivity tested using EGFP lentivirus (MOI=5) to infect HEK293T cells (25,000 cells) in the presence of 2.5% HPBCD formulated with zinc and ascorbic acid was unable to prevent lentivirus infection when incubated for 0.5 and 2 hours. Both 5% and 10% HPBC formulated with Zn and ascorbic acid was able to reduce viral infectivity after 0.5 hours of incubation but not at 2 hours. 10% HPBCD formulation contains 3 mg/mL of Zn and 9 mg/mL of ascorbic acid. Other formulations were serially diluted from the 10% HPBCD+additive formulation.

FIG. 11. The infectivity of viruses is inhibited when lentivirus was incubated with HPBCD nano formulations that has zinc and ascorbic acid additives. Viral transmissivity was tested using EGFP lentivirus (MOI=5) to infect HEK293T cells (25,000 cells). The lentivirus was incubated for an hour with HPBCD formulated with zinc and ascorbic acid. An aliquot of the mix was added unto cells to evaluate infectivity. Results showed significant reduction in the ability of the lentivirus to infect cells after incubation with the barrier. The 10% HPBCD formulation contains 3 mg/mL of Zn and 9 mg/mL of ascorbic acid. Other formulations were serially diluted from the 10% HPBCD+additive formulation.

FIG. 12. The effect of various nanoformulation barriers on A549 cell morphology. No significant changes in cellular morphology or cell death were observed in HPBCD barriers with and without HEC. However, A549 cellular death and cellular morphological changes were seen in CRYSMEB barrier. This affirms the fact that not every cyclodextrin derivative is safe for these types of application.

FIG. 13. In silico 3D modeling of the nasopharynx. Exploration of parameter space using a 3D model and particle size less than 10 microns (A) the effect of penetration depth upon particle deposition in the nasal cavity (B) the effect of spray angle on particle deposition (C) the effect of degrees of anterior block on decrease in particle deposition.

FIG. 14. In vitro HPBCD barrier studies. The assessment of barrier efficacy in reducing chemical and viral penetration (A) dye penetration in the presence of HPBCD as measured by absorbance (B) viral infectivity in the presence of HPBCD as indicated by GFP.

### DETAILED DESCRIPTION

### Definitions

The following definitions are included to provide a clear and consistent understanding of the specification and claims. As used herein, the recited terms have the following meanings. All other terms and phrases used in this specification have their ordinary meanings as one of skill in the art would understand. Such ordinary meanings may be obtained by reference to technical dictionaries, such as Hawley's Condensed Chemical Dictionary 14^{th} Edition, by R. J. Lewis, John Wiley & Sons, New York, N.Y., 2001.

Reference herein to “one embodiment”, “an embodiment”, etc., indicate that the embodiment described may include a particular aspect, feature, structure, moiety, or characteristic, but not every embodiment necessarily includes that aspect, feature, structure, moiety, or characteristic. Moreover, such phrases may, but do not necessarily, refer to the same embodiment referred to in other portions of the specification. Further, when a particular aspect, feature, structure, moiety, or characteristic is described in connection with an embodiment, it is within the knowledge of one skilled in the art to connect such aspect, feature, structure, moiety, or characteristic with other embodiments, whether explicitly described.

The singular forms “a,” “an,” and “the” include plural reference unless the context clearly dictates otherwise. Thus, for example, a reference to “a compound” includes a plurality of such compounds, so that a compound X includes a plurality of compounds X. It is further noted that the claims may be drafted to exclude any optional element. As such, this statement is intended to serve as antecedent basis for the use of exclusive terminology, such as “solely,” “only,” and the like, in connection with any element described herein, and/or the recitation of claim elements or use of “negative” limitations.

The term “and/or” means any one of the items, any combination of the items, or all the items with which this term is associated. The phrases “one or more” and “at least one” are readily understood by one of skill in the art, particularly when read in context of its usage. For example, the phrase can mean one, two, three, four, five, six, ten, 100, or any upper limit approximately 10, 100, or 1000 times higher than a recited lower limit.

As will be understood by the skilled artisan, all numbers, including those expressing quantities of ingredients, properties such as molecular weight, reaction conditions, and so forth, are approximations and are understood as being optionally modified in all instances by the term “about.” These values can vary depending upon the desired properties sought to be obtained by those skilled in the art utilizing the teachings of the descriptions herein. It is also understood that such values inherently contain variability necessarily resulting from the standard deviations found in their respective testing measurements. When values are expressed as approximations, by use of the antecedent “about,” it will be understood that the particular value without the modifier “about” also forms a further aspect.

The terms “about” and “approximately” are used interchangeably. Both terms can refer to a variation of ±2.5%, ±5%, ±10%, ±20%, or ±25% of the value specified. For example, “about 50” percent can in some embodiments carry a variation from 45 to 55 percent, or as otherwise defined by a particular claim. For integer ranges, the term “about” can include one or two integers greater than and/or less than a recited integer at each end of the range. Unless indicated otherwise herein, the terms “about” and “approximately” are intended to include values, e.g., weight percentages, proximate to the recited range that are equivalent in terms of the functionality of the individual ingredient, composition, or embodiment. The terms “about” and “approximately” can also modify the endpoints of a recited range as discussed above in this paragraph.

As will be understood by one skilled in the art, for any and all purposes, particularly in terms of providing a written description, all ranges recited herein also encompass any and all possible sub-ranges and combinations of sub-ranges thereof, as well as the individual values making up the range, particularly integer values. It is therefore understood that each unit between two particular units are also disclosed. For example, if 10 to 15 is disclosed, then 11, 12, 13, and 14 are also disclosed, individually, and as part of a range. A recited range (e.g., weight percentages or carbon groups) includes each specific value, integer, decimal, or identity within the range. Any listed range can be easily recognized as sufficiently describing and enabling the same range being broken down into at least equal halves, thirds, quarters, fifths, or tenths. As a non-limiting example, each range discussed herein can be readily broken down into a lower third, middle third and upper third, etc. As will also be understood by one skilled in the art, all language such as “up to”, “at least”, “greater than”, “less than”, “more than”, “or more”, and the like, include the number recited and such terms refer to ranges that can be subsequently broken down into sub-ranges as discussed above. In the same manner, all ratios recited herein also include all sub-ratios falling within the broader ratio. Accordingly, specific values recited for radicals, substituents, and ranges, are for illustration only; they do not exclude other defined values or other values within defined ranges for radicals and substituents. It will be further understood that the endpoints of each of the ranges are significant both in relation to the other endpoint, and independently of the other endpoint.

One or ordinary skill in the art will also readily recognize that where members are grouped together in a common manner, such as in a Markush group, the invention encompasses not only the entire group listed as a whole, but each member of the group individually and all possible subgroups of the main group. Additionally, for all purposes, the invention encompasses not only the main group, but also the main group absent one or more of the group members. The invention therefore envisages the explicit exclusion of any one or more of members of a recited group. Accordingly, provisos may apply to any of the disclosed categories or embodiments whereby any one or more of the recited elements, species, or embodiments, may be excluded from such categories or embodiments, for example, for use in an explicit negative limitation.

The term “contacting” refers to the act of touching, making contact, or of bringing to immediate or close proximity, including at the cellular or molecular level, for example, to bring about a physiological reaction, a chemical reaction, or a physical change, e.g., in a solution, in a reaction mixture, in vitro, or in vivo.

An “effective amount” refers to an amount effective to treat a disease, disorder, and/or condition, or to bring about a recited effect. For example, an effective amount can be an amount effective to reduce the progression or severity of the condition or symptoms being treated. Determination of a therapeutically effective amount is well within the capacity of persons skilled in the art. The term “effective amount” is intended to include an amount of a compound described herein, or an amount of a combination of compounds described herein, e.g., that is effective to treat or prevent a disease or disorder, or to treat the symptoms of the disease or disorder, in a host. Thus, an “effective amount” generally means an amount that provides the desired effect.

Alternatively, the terms “effective amount” or “therapeutically effective amount,” as used herein, refer to a sufficient amount of an agent or a composition or combination of compositions being administered which will relieve to some extent one or more of the symptoms of the disease or condition being treated. The result can be reduction and/or alleviation of the signs, symptoms, or causes of a disease, or any other desired alteration of a biological system. For example, an “effective amount” for therapeutic uses is the amount of the composition comprising a compound as disclosed herein required to provide a clinically significant decrease in disease symptoms. An appropriate “effective” amount in any individual case may be determined using techniques, such as a dose escalation study. The dose could be administered in one or more administrations. However, the precise determination of what would be considered an effective dose may be based on factors individual to each patient, including, but not limited to, the patient's age, size, type or extent of disease, stage of the disease, route of administration of the compositions, the type or extent of supplemental therapy used, ongoing disease process and type of treatment desired (e.g., aggressive vs. conventional treatment).

The terms “treating”, “treat” and “treatment” include (i) preventing a disease, pathologic or medical condition from occurring (e.g., prophylaxis); (ii) inhibiting the disease, pathologic or medical condition or arresting its development; (iii) relieving the disease, pathologic or medical condition; and/or (iv) diminishing symptoms associated with the disease, pathologic or medical condition. Thus, the terms “treat”, “treatment”, and “treating” can extend to prophylaxis and can include prevent, prevention, preventing, lowering, stopping or reversing the progression or severity of the condition or symptoms being treated. As such, the term “treatment” can include medical, therapeutic, and/or prophylactic administration, as appropriate.

As used herein, “subject” or “patient” means an individual having symptoms of, or at risk for, a disease or other malignancy. A patient may be human or non-human and may include, for example, animal strains or species used as “model systems” for research purposes, such a mouse model as described herein. Likewise, patient may include either adults or juveniles (e.g., children). Moreover, patient may mean any living organism, preferably a mammal (e.g., human or non-human) that may benefit from the administration of compositions contemplated herein. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish and the like. In one embodiment of the methods provided herein, the mammal is a human.

As used herein, the terms “providing”, “administering,” “introducing,” are used interchangeably herein and refer to the placement of the compositions of the disclosure into a subject by a method or route which results in at least partial localization of the composition to a desired site. The compositions can be administered by any appropriate route which results in delivery to a desired location in the subject.

The compositions described herein may be administered with additional compositions to prolong stability and activity of the compositions, or in combination with other therapeutic drugs.

The terms “inhibit”, “inhibiting”, and “inhibition” refer to the slowing, halting, or reversing the growth or progression of a disease, infection, condition, or group of cells. The inhibition can be greater than about 20%, 40%, 60%, 80%, 90%, 95%, or 99%, for example, compared to the growth or progression that occurs in the absence of the treatment or contacting.

The term “substantially” as used herein, is a broad term and is used in its ordinary sense, including, without limitation, being largely but not necessarily wholly that which is specified. For example, the term could refer to a numerical value that may not be 100% the full numerical value. The full numerical value may be less by about 1%, about 2%, about 3%, about 4%, about 5%, about 6%, about 7%, about 8%, about 9%, about 10%, about 15%, or about 20%.

### Embodiments of the Disclosure

The emergence and re-emergence of pathogenic viruses is occurring at an unprecedented rate causing concerns for global health concerns. The high infectivity and transmissibility of viral pathogens such as the novel SARS-CoV-2 that leads to severe pulmonary sequelae has highlighted the need for effective preventive modalities. The ability to prevent viral attachment and cellular entry through disrupting the interactions between viral proteins with tropic factors found on the surface of epithelial cell membranes may be an effective approach.

Further, highly toxic chemical agents and transmittable pathogens can be aerosolized and inhaled through mucocutaneous membranes of the body's passages. The ability to prevent chemical contact and viral attachment onto mucocutaneous membranes may be an effect approach to prevent toxic chemicals as well as highly infective and transmittable pathogens. Here we present data in support of cyclodextrin-based formulation as nanoscale barriers to prevent chemical exposure and viral infection. To expedite on development, we employed an in silico computational models to identify physicochemical parameters to guide the development of nanoscale products.

Cyclodextrins (CDs) and in particular, hydroxypropyl beta cyclodextrins (HPBCD), 2-Hydroxypropyl-Gamma-cyclodextrin (HPGCD) crystalline methylated beta-cyclodextrin (CRYSMEB) and sulfobutyl ether-beta-cyclodextrin (SBEBCD), are widely used as solubilizing agents, stabilizers, and inert excipients in pharmaceutical compositions (see U.S. Pat. Nos. 6,194,430; 6,194,395; and 6,191,137, each of which is incorporated herein by reference). HPBCDs are cyclic compounds containing seven units of a-(1, 4) linked D-glucopyranose units, and act as complexing agents that can form inclusion complexes and have concomitant solubilizing properties (see U.S. Pat. No. 6,194,395; see, also, Szejtli, J. Cyclodextrin Technol, 1988). As disclosed herein, HPBCD or another disclosed cyclodextrin may block entrance of a viral pathogen through the membrane of a susceptible cell by forming a barrier between the cell and the viral pathogen, disrupting the lipid rafts in cell membrane be sequestering cholesterol from the host cell, and by sequestering cholesterol from the membrane of the viral pathogen.

Certain embodiments of the compositions and methods of the invention may be exemplified by the use of 2-hydroxypropyl-β-cyclodextrin (2-hydroxypropyl-BCD). However, any BCD derivative can be used in a composition, formulation, or method of the invention, provided the BCD derivative disrupts lipid rafts in the membranes of cells susceptible to a viral pathogen by removal of cholesterol from the host cell membrane without causing undesirable side effects. BCDs are variably effective in such removal. For example, methyl-BCD removes cholesterol from cell membranes very efficiently and quickly and, as a result, can be toxic to cells, which require cholesterol for membrane integrity and viability. In comparison, a BCD derivative such as 2-hydroxypropyl-BCD may effectively remove cholesterol from cells without producing undue toxicity.

BCDs useful in the present invention include, for example, BCD derivatives wherein one or more of the hydroxy groups is substituted by an alkyl, hydroxyalkyl, carboxyalkyl, alkylcarbonyl, carboxyalkoxyalkyl, alkylcarbonyloxyalkyl, alkoxycarbonylalkyl or hydroxy-(mono or polyalkoxy)alkyl group or the like; and wherein each alkyl or alkylene moiety contains up to about six carbons. Substituted BCDs that can be used in the present invention include, for example, polyethers (see, for example, U.S. Pat. No. 3,459,731, which is incorporated herein by reference); ethers, wherein the hydrogen of one or more BCD hydroxy groups is replaced by C1 to C6 alkyl, hydroxy-C1-C6-alkyl, carboxy-C1-C6 alkyl, C1-C6 alkyloxycarbonyl-C1-C6 alkyl groups, or mixed ethers thereof. In such substituted BCDs, the hydrogen of one or more BCD hydroxy group can be replaced by C1-C3 alkyl, hydroxy-C2-C4 alkyl, or carboxy-C1-C2 alkyl, for example, by methyl, ethyl, hydroxyethyl, hydroxypropyl, hydroxybutyl, carboxymethyl, or carboxyethyl. It should be recognized that the term “C1-C6 alkyl” includes straight and branched saturated hydrocarbon radicals, having from 1 to 6 carbon atoms. Examples of BCD ethers include dimethyl-BCD. Examples of CD polyethers include hydroxypropyl-p-BCD and hydroxyethyl-BCD (see, for example, U.S. Pat. Nos. 3,459,731; 4,535,152; WO90/12035; GB-2,189,245; U.S. Pat. Nos. 5,134,127A; 6,194,395, 4,659,696, and 4,383,992, each of which is incorporated herein by reference). Preferably, the CD comprises about 2.5%, about 5%, about 10%, about 15%, or about 20% by weight of the formulation.

Further, HPBCD or other CDs can facilitate removal of some of the host cellular membrane cholesterol molecules, thus rendering them less susceptible to viral infection. HPBCD can bind onto a wide spectrum of viruses, like human immunodeficiency virus (HIV), human metapneumovirus (HMPV), parainfluenza virus type 3 (HPIV3), Influenza virus A-D, coronavirus infectious bronchitis virus (IBV), herpes simplex virus 1 (HSV-1), herpes simplex II (HSV-2) varicella-zoster virus (VZV), cytomegalovirus (CMV), Epstein Bane virus (EBV), Kaposi's Sarcoma herpes virus (KSHS), hepatitis A virus (HAV), hepatitis C virus (HCV), hepatitis B virus, human papilloma virus (HPV), SARS-CoV-1, MERS virus, and SARS-CoV-2 virus and its variants (e.g., B.1.1.7, B.1.351, B.1.525, B.1.617, B.1.429, B.1.427, B.1.1.207, and P.1). However, we are proposing a novel HPBCD-based platform formulation capable of preventing viral attachment without compromising the antiviral potency.

In other embodiments, the cyclodextrin formulations prevent contact and entry of a bacteria into a cell to which the coating is applied. Exemplary bacteria include, but are not limited to, Achromobacter sp., Acinetobacter sp., Actinomyces sp., Aeromonas sp., Bacillus sp., Bacteroides sp., Bartonella sp., Bordetella sp., Borrelia sp., Brucella sp., Burkholderia sp., Campylobacter sp., Chlamydophila sp., Clostridium species sp., Ehrlichia sp., Enterobacter sp., Enterococcus sp., Escherichia sp., Haemophilus sp., Helicobacter sp., Klebsiella sp., Lactobacillus sp., Legionella sp., Mycoplasma sp., Neisseria sp., Nocardia sp., Pseudomonas sp., Salmonella sp., Shigella sp., Staphylococcus sp., Streptococcus sp., and Vibrio sp.

In one embodiment, a cyclodextrin is formulated as a nano-formulation for reducing the risk of a microbial infection comprising a cyclodextrin present in a concentration of about 5% to about 20% by weight of the nano-formulation, wherein the cyclodextrin is one or more selected from the group consisting of hydroxypropyl-beta-cyclodextrin, 2-hydroxypropyl-gamma-cyclodextrin, crystalline methylated-beta-cyclodextrin, sulfobutyl-ether-beta-cyclodextrin, and a pharmaceutically acceptable carrier.

In one embodiment, a cyclodextrin is formulated as a nano-formulation for reducing the risk of a microbial infection comprising a cyclodextrin present in a concentration of about 2.5% to about 20% by weight of the nano-formulation, wherein the cyclodextrin is one or more selected from the group consisting of hydroxypropyl-beta-cyclodextrin, 2-hydroxypropyl-gamma-cyclodextrin, crystalline methylated-beta-cyclodextrin, sulfobutyl-ether-beta-cyclodextrin, and a pharmaceutically acceptable carrier, wherein the formulation comprises 0.9% (w/v) or less of zinc or zinc-containing compounds.

In one embodiment, a cyclodextrin is formulated as a nano-formulation for reducing the risk of a microbial infection comprising a cyclodextrin present in a concentration of about 2.5% to about 20% by weight of the nano-formulation, wherein the cyclodextrin is one or more selected from the group consisting of hydroxypropyl-beta-cyclodextrin, 2-hydroxypropyl-gamma-cyclodextrin, crystalline methylated-beta-cyclodextrin, sulfobutyl-ether-beta-cyclodextrin, a thickening agent, and/or another a pharmaceutically acceptable carrier.

In another embodiment, a cyclodextrin is formulated as an aerosolized particulate nano-formulation for reducing the risk of a microbial infection consisting of a cyclodextrin present in a concentration of about 2.5% to about 20% by weight of the nano-formulation, wherein the cyclodextrin is one or more selected from the group consisting of hydroxypropyl-beta-cyclodextrin, 2-hydroxypropyl-gamma-cyclodextrin, crystalline methylated-beta-cyclodextrin, sulfobutyl-ether-beta-cyclodextrin, a thickening agent, wherein the aerosolized particulate has a mean particle diameter of up to about 100 μm in diameter, and a pharmaceutically acceptable carrier in other dosage forms.

In some embodiments, a cyclodextrin or formulation comprising cyclodextrin as described herein may be formulated into a nasal, mouth or eye application (including the forms of nasal spray, eye drops, mouth wash, nasal gel, mouth gel, eye gel). Preferred embodiments can be used in the form of inhalation solution, pressurized aerosol, eye drops or nasal drops, and in a particular preferred embodiment, in the form of a spray (preferably a nasal spray). A spray can, for example, be formed by the use of a conventional spray-squeeze bottle or a pump vaporizer.

Some embodiments may use compressed gas aerosols. Suitable propellants for use in compressed gas aerosols include 1,1,1,2-tetrafluoroethane (HFA 134a) or 1,1,1,2,3,3,3,-heptafluoropropane (HFA 227), or a combination of both, or mono-fluoro trichloromethane and dichloro difluoromethane, in particular 1,1,1,2-tetrafluoroethane (HFA 134a) or 1,1,1,2,3,3,3-heptafluoropropane (HFA 227).

In some embodiments, a formulation may include a preservative and/or a stabilizer. These may include, for example, ethylene diamine tetra-acetic acid (EDTA) and its alkali salts (for example dialkali salts such as disodium salt, calcium salt, calcium-sodium salt), lower alkyl p-hydroxybenzoates, chlorhexidine (for example in the form of the acetate or gluconate) and phenyl mercury borate. Other suitable preservatives include pharmaceutically useful quaternary ammonium compounds, for example cetylpyridinium chloride, tetradecyltrimethyl ammonium bromide, benzyldimethyl-[2-[2[p-(1,1,3,3-tetramethyl-butyl)phenoxy]ethoxy]-ammonium chloride i.e., benzethonium chloride, and myristyl picolinium chloride. Generally, each preservative or stabilizer may be used in a concentration of about 0.002% to about 0.05%, for example 0.02% (weight/volume in liquid formulations, otherwise weight/weight). The total amount of preservatives in the formulations (solutions, ointments, etc.) preferably is from about 0.001 g to about 0.10 g, and preferably about 0.01 g per 100 ml of solution/suspension or 100 g of formulation.

Other exemplary amounts of preservatives include thimerosal from about 0.002% to about 0.02%; benzalkonium chloride from about 0.002% to about 0.02%; chlorhexidine acetate or gluconate from about 0.01% to about 0.02%; phenyl mercuric/nitrate, borate, acetate from about 0.002% to about 0.004%; p-hydroxybenzoic acid ester from about 0.05% to about 0.15%, or more preferably, about 0.1%.

Other substances that may be used in a formulation of the disclosure include polyvinyl pyrrolidone, sorbitan fatty acid esters such as sorbitan trioleate, polyethoxylated sorbitan fatty acid esters (for example polyethoxylated sorbitan trioleate), sorbimacrogol oleate, synthetic amphotensides (tritons), ethylene oxide ethers of octylphenolformaldehyde condensation products, phosphatides such as lecithin, polyethoxylated fats, polyethoxylated oleotriglycerides and polyethoxylated fatty alcohols. In this context, polyethoxylated means that the relevant substances contain polyoxyethylene chains, the degree of polymerization of which is generally between 2 to 40, in particular between 10 to 20.

Some embodiments also may include one or more isotonization agents. Isotonization agents which may, for example, adjust the osmotic pressure of the formulations to the same osmotic pressure, for example, as nasal secretion. Exemplary isotonization agents include saccharose, glucose, glycerine, sorbitol, 1,2-propylene glycol and NaCl.

Some embodiments of the disclosure also may include one or more pharmaceutically acceptable carriers and/or excipients. In some embodiments, the pharmaceutically acceptable carrier is water or distilled water. In other embodiments, the pharmaceutically acceptable carrier is a thickening agent. The thickening agent may be used, for example, to prevent the solution from flowing out of the nose too quickly. In some embodiments, the thickener may give the formulation a viscosity of at least 1.5 mPa, and preferably 2 mPa.

Exemplary thickening agents may include cellulose polymers, cellulose derivatives (for example cellulose ether) in which the cellulose-hydroxy groups are partially etherified with lower unsaturated aliphatic alcohols and/or lower unsaturated aliphatic oxyalcohols (for example methyl cellulose, carboxymethyl cellulose, hydroxypropylmethylcellulose), gelatin, polyvinylpyrrolidone, tragacanth, ethoxose (water soluble binding and thickening agents on the basis of ethyl cellulose), alginic acid, polyvinyl alcohol, polyacrylic acid, pectin, poloxamers (tri-block copolymers characterized by the presence of hydrophobic poly(propylene oxide) (PPO) between two blocks of hydrophilic poly(ethylene oxide) (PEO)) and equivalent agents. Should these substances contain acid groups, the corresponding physiologically acceptable salts may also be used. In some embodiments, the thickening agent is hydroxyethyl cellulose or methyl cellulose.

In some embodiments, the thickening agent in a formulation is present in a ratio of cyclodextrin to thickening agent of about 5:1, of about 10:1, of about 11:1, of about 12:1, of about 13:1, of about 14:1, or about 15:1. In some embodiments, the ratio of cyclodextrin to thickening agent in the formulation is about 13:1 or 13:1. In another embodiment, the ratio of cyclodextrin to thickening agent of about 5:3. In another embodiment, the ratio of cyclodextrin to thickening agent of about 5:3 to about 15:1.

In some embodiments, the mean particulate size diameter of the nanoformulations whose aerosolized particulates may range from about 10 μm to about 150 μm, about 15 μm to about 125 μm, about 20μm to about 100 μm, about 25 μm to about 75 μm, about 35 μm to about 65 μm, about 45 μm to about 55 μm, or about 50 μm.

In some embodiments, a cyclodextrin containing formulation excludes zinc or a zinc containing compounds. In other embodiments, the formulations comprise 0.9% (w/v) or less of zinc or zinc-containing compound. For example, a formulation may comprise about 0.1% or less, about 0.2% or less, about 0.3% or less, about 0.4% or less, about 0.5% or less, about 0.6% or less, about 0.7% or less, about 0.8% or less, about 0.9% or less, or about 0.9% zinc or zinc containing compounds.

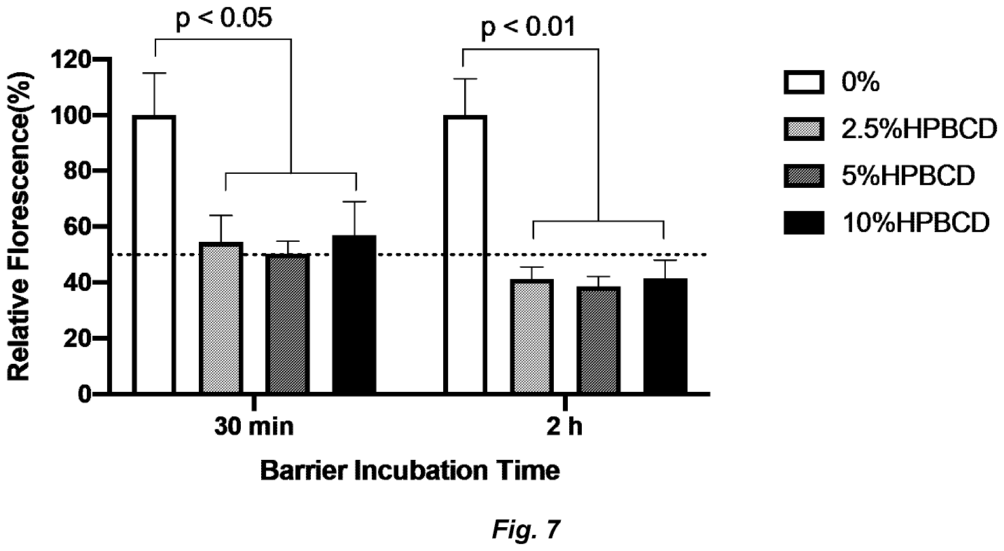

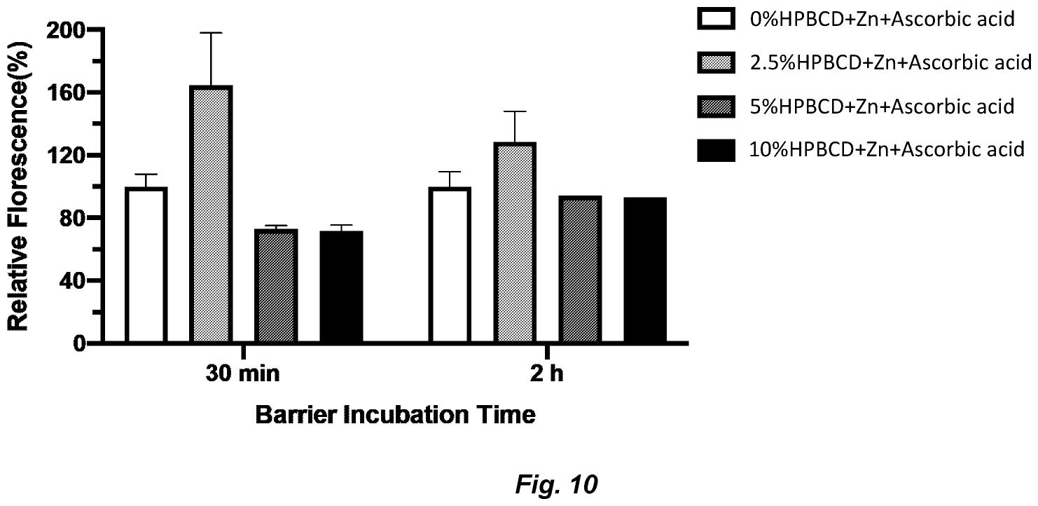

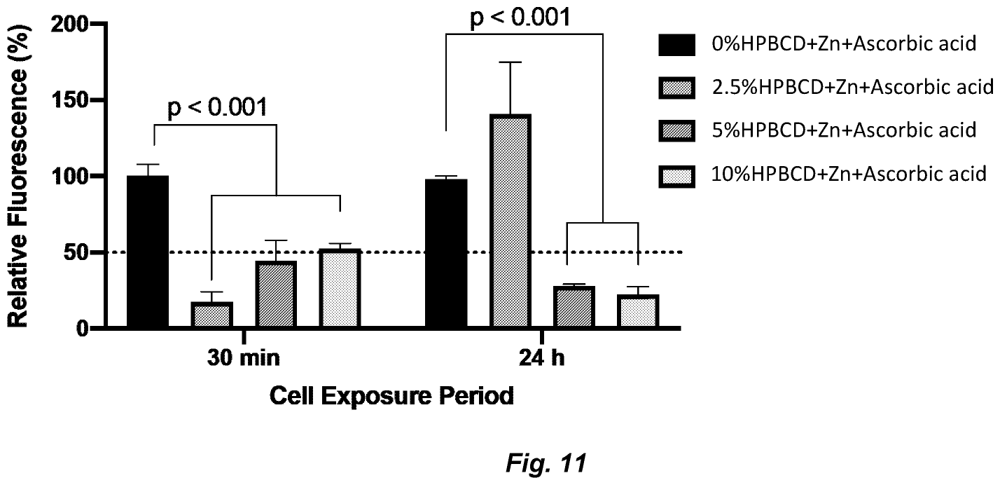

It has been shown that zinc compounds, such as zinc oxide and zinc pyrithione, have antibacterial and antifungal activities. For example, zinc oxide has been used in antibacterial creams, anti-rash creams, and other medical remedies. Zinc oxide also is known to have antiviral activities and has been used in a variety of formulations to treat or prevent viral infections. For example, U.S. Pat. No. 6,638,915 describes a method of making and using a mixture containing a combination of zinc oxide, aspartic acid, and high fructose corn syrup, as an antiviral remedy. In contrast, Applicants have unexpectedly found that concentrations of zinc or zinc containing compounds greater than 0.9% (w/v) reduces the efficacy of the cyclodextrin formulations (compare FIG. 7 and FIG. 10) in preventing infectivity when applied to a mucosal membrane. Interestingly though, the presence of zinc and/or ascorbic acid inhibited viral transmission when viral particles were incubated with the cyclodextrin formulation comprising zinc and ascorbic acid (FIG. 11).

Certain embodiments of the disclosure also provide for a method of reducing a risk of a microbial infection comprising contacting cells susceptible to infection with an effective amount of a formulation as described herein to form a barrier, wherein the barrier prevents a microbe from contacting the cells, thereby reducing the risk of the microbial infection.

Preferably, the method includes the use of an aerosol, inhalant, intranasal spray, or an aqueous spray, wherein the formulation comprises the use of a specialized device to contact the cells susceptible to infection with the formulation.

In some embodiments of a method of the disclosure, the specialized device is spray device having a dispensing nozzle that may be positioned at a certain angle and nozzle depth in, for example, the nostril, to maximize distribution of the cyclodextrin formulation to the mucosal membranes of the nasopharynx. In some embodiments, the formulation is deposited in the target using a spray cone angle of about π/2, about π/3, about π/4, or about π/6. In some embodiments, the spray cone angle is π/2.

In some embodiments, the insertion depth of the dispensing nozzle or equivalent devices into the target location (e.g., nostril) is about 1 mm, or about 2 mm, or about 3 mm, or about 4 mm, or about 5 mm, or about 6 mm, or about 7 mm, or about 8 mm, or about 9 mm or about 10 mm, or about 11 mm, or about 12 mm, or about 13 mm, or about 14 mm, or about 15 mm.

In some embodiments, the sprayed volume applied to a target location is about 10 μl, about 20 μl, about 30 μl, about 40 μl, about 50 μl, about 60 μl, about 70 μl, about 80 μl, about 90 μl, about 100 μl, about 110 μl, about 120 about 130 μl, about 140 μl, about 150 μl.

In some embodiments, the velocity of the sprayed volume is about 1 m/s, about 2 m/s, about 3 m/s, about 4 m/s, about 5 m/s, about 6 m/s, about 7 m/s, about 8 m/s, about 9 m/s, about 10 m/s, 11 m/s, about 12 m/s, about 13 m/s, about 14 m/s, about 15 m/s, about 16 m/s, about 17 m/s, about 18 m/s, about 19 m/s, or about 20 m/s.

In one embodiment, volume sprayed is about 50 μl to 80 μl, the spray velocity is about 4 m/s, the insertion depth is about 10 mm, and the spray cone angle is about π/2.

In another embodiment, the volumed sprayed is about 50 μl to 80 μl, the spray velocity is about 18 m/s, insertion depth is about 10 mm, and a spray cone angle is about π/3.

In some embodiments, the formulation is an aerosolized particulate.

In another embodiments, an aerosolized particulate may be delivered at a spray volume of about 50 μl to 160 μl, a spray velocity of about 4 m/s to 18 m/s, an insertion depth of about 10 mm, and a spray cone angle of about π/4 to about π/2.

In some embodiments, the nanoformulation is applied to a mucocutaneous lining of a target area. In some embodiments, the mucocutaneous lining is a surface of one or more of a nasal cavity, an oropharyngeal cavity, a gastrointestinal cavity, a bronchial cavity, a pulmonary cavity, an oral cavity, a rectal cavity, a vaginal cavity, or an ocular cavity.

In some embodiments, the formulation is delivered to a target area greater than 1.5 mm from an entry point of the cavity comprising the mucocutaneous linings (e.g., the entry point is the nostrils, mouth, rectal opening, vaginal opening, etc.).

### Results and Discussion

### 3D Modeling of Spray Characteristics

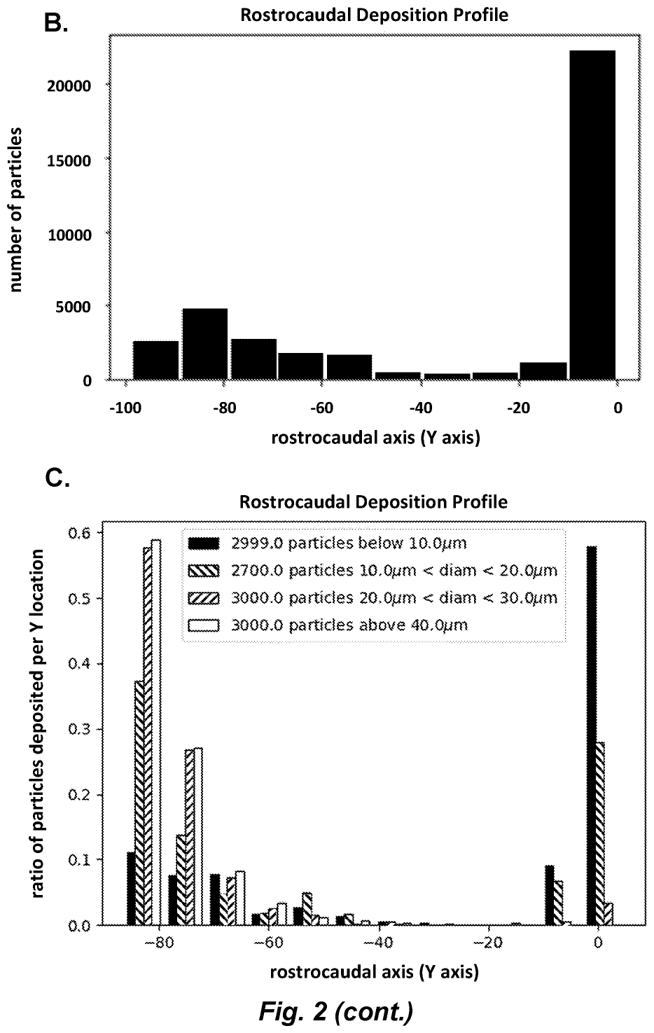

To accelerate the design of a delivery device and barrier formulation, we developed and used in silico modeling using published and calculated parameters. The computational models and methodologies developed within this framework are aimed to guide the overall development at various stages. Initial models were developed using established data showing that the nasal passage was the most susceptible site of viral infections like SARS-CoV-2 infections. The deposition profile obtained with SARS-CoV-2 virally charged particles of different sizes confirmed results obtained concurrently by multiple groups that indicate that larger-sized particles tend to deposit on the walls of the nasal cavity, thereby increasing the risk of infectivity as they may interact with nasal epithelial cells, cells that have been shown to be the most prone to SARS-CoV-2 infectivity. On the other end, the major portion of smaller particles tend to exit the nasal cavity and enter the lower portion of the respiratory tract (FIG. 2).

The inputs into the model included particle size, potential inhalation of these particles, using iterative design approach. Using the same computational fluid dynamics (CFD) methodologies to model viral deposition, we superimpose these parameters to construct delivery of aerosolized cyclodextrin nano formulation particles to form an optimal barrier. Input parameters such as aerosolized particle size, diameter of spray cone and spray cone angle on particle deposition patterns in a realistic human model of the upper respiratory tract.

### Effect of Particle Distribution Function

The model indicated that mean aerosolized particle size used for the nanoformulation particle distribution function has a drastic effect on deposition. With all parameters equal, our results indicate that for the same volume of cyclodextrin delivered, we obtain a drastically better coverage with smaller particles than with larger ones. Decreasing the mean particle diameter from 100 μm to 50 μm (using a lognormal distribution function and a spraying volume of 80 μl) improved coverage of the nasal cavity walls from 8% to 15%. This significant improvement in deposition is due to a better distribution of smaller particles as well as a higher number of small particles for a given volume of HPBCD sprayed.

### Product Target Profile

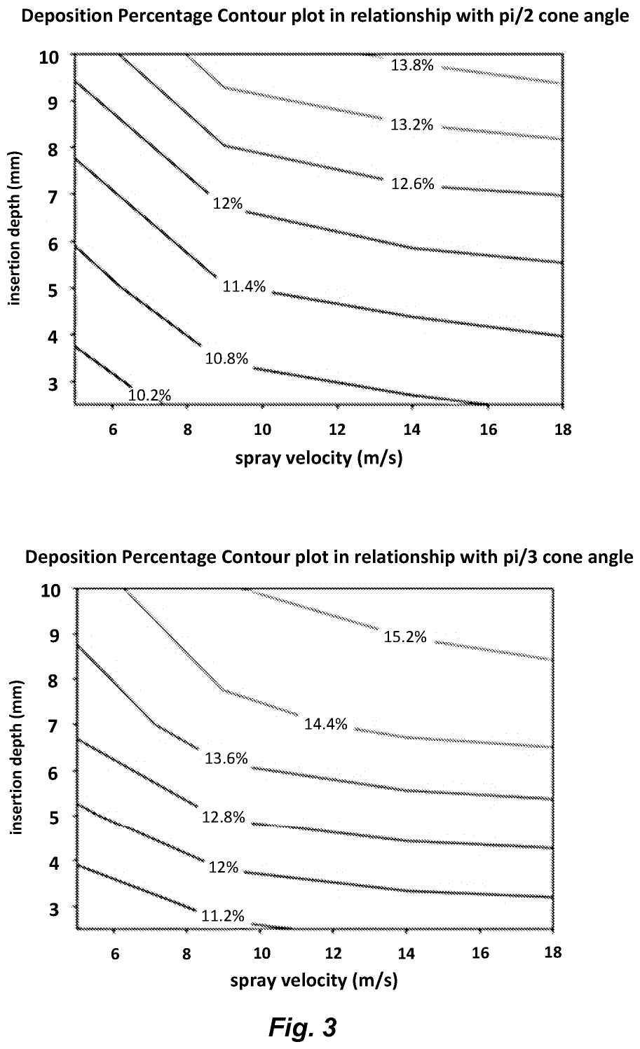

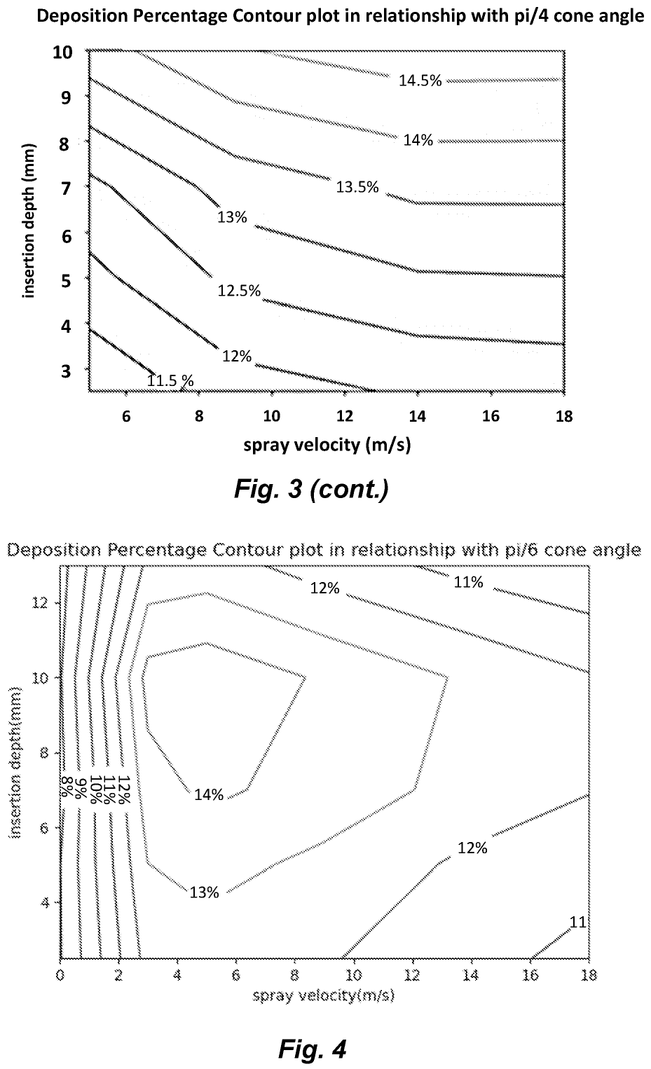

To establish an optimal product target profile, large numbers of simulations were performed while systematically varying spray parameter values for spray head insertion depth, spray cone angle and spray velocity with a log-normal particle-size distribution (PSD)(Kundoor et al., Pharmaceutical research 28.8 (2011): 1895-1904) profile of particle. This allowed us to (i) obtain a good understanding of the contribution of each parameter on the deposition profile, and (ii) determine the optimal range of parameters. An example of results obtained is presented in FIG. 3, with a mean spray particle diameter of 50 μm on a lognormal probability density function (PDF); number of particles is 300,000; sprayed volume is 80 μl. For these parameters, optimal profile was obtained with a spray velocity of 18 m/s, insertion depth of 10 mm and a spray cone angle of π/3. Notably, all simulations were run with parameters varying within the realistic range (volume/actuation: 50 μl to 140 μl (Sungnak et al., Nature Medicine, 2020. 26(5): p. 681-687.), mean particle diameter: 20 μm to 100 μm (Kundoor et al., Pharmaceutical research 28.8 (2011): 1895-1904; Basu et al., Scientific reports 10, no. 1 (2020): 1-18.)) spray velocity: 6 to 18 m/s, spray cone angle: 30 to 60 degrees (Thrall et al., Inhalation Toxicology, 2003. 15(6): p. 523-538) of values reported in different commercial spraying applications (FIG. 3).

### Incorporation of Inhalation Speed

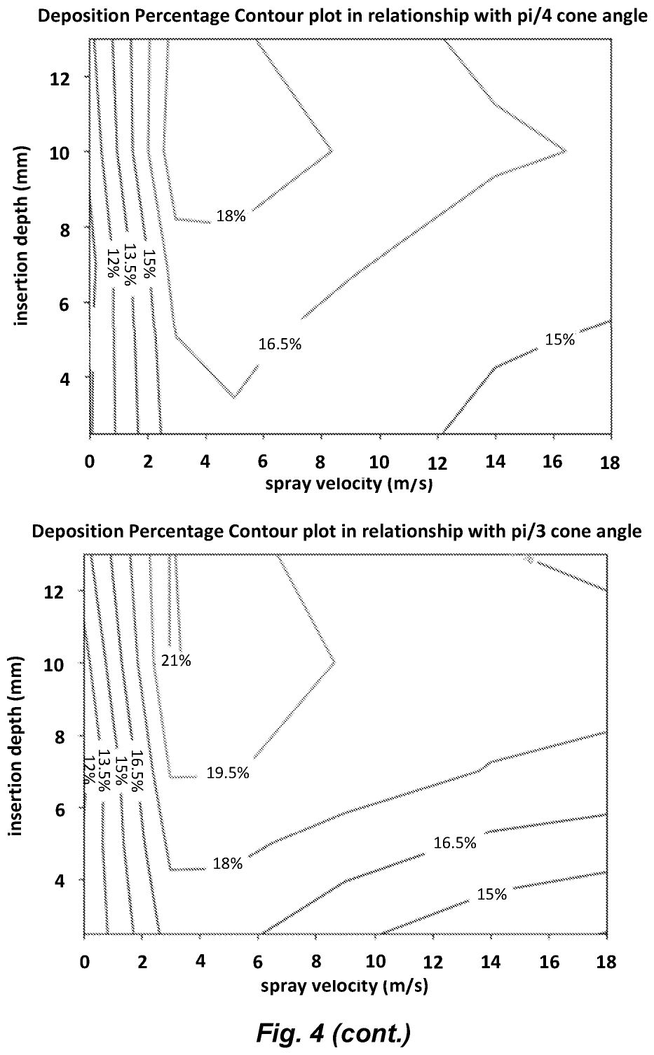

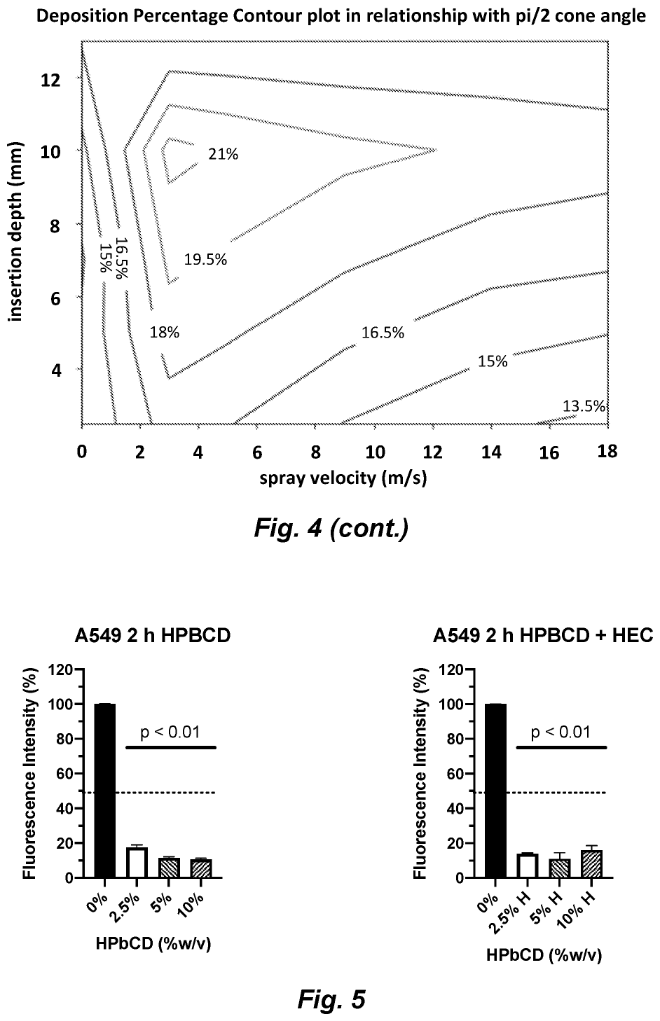

Results from our CFD simulations indicated that smaller-sized particles tend to deposit at the bottom of the nasal cavity (also visible in FIG. 2A). Incorporating in our simulations an inhalation airflow in conjunction with the spray velocity resulted in an improvement in overall coverage (FIG. 4). Of importance, the systematic parameter exploration also outlines the sensitivity of deposition to variations (intentional or not) in parameter values. For example, the computational results in FIG. 4 indicate the consequence of the user not inserting the spray to the optimal 10 mm mark. Consequently, the computational approach not only facilitates identification of optimal parameter values, but also outline an acceptable therapeutic window.

### Efficacy of Barrier in Preventing Chemical Penetration

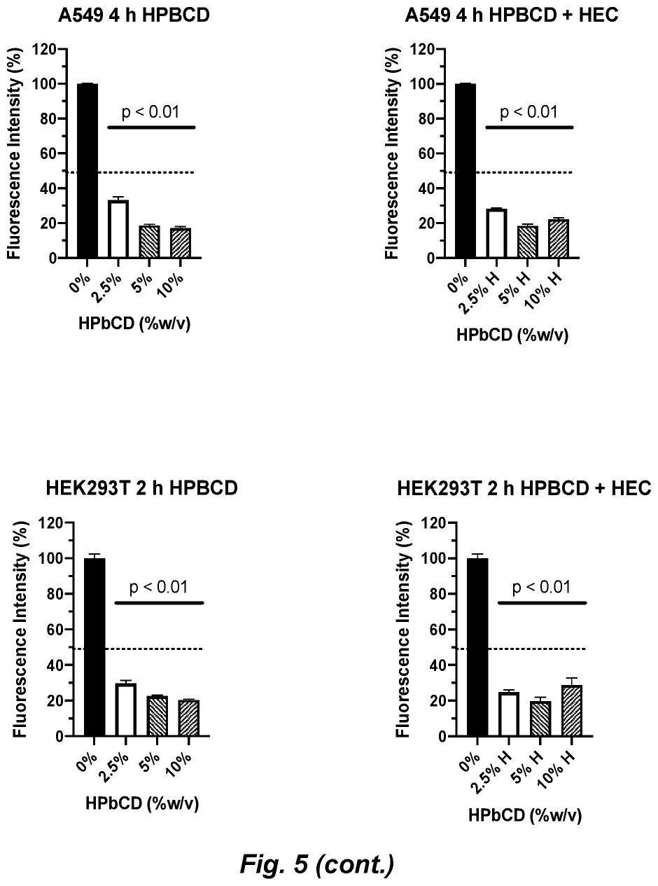

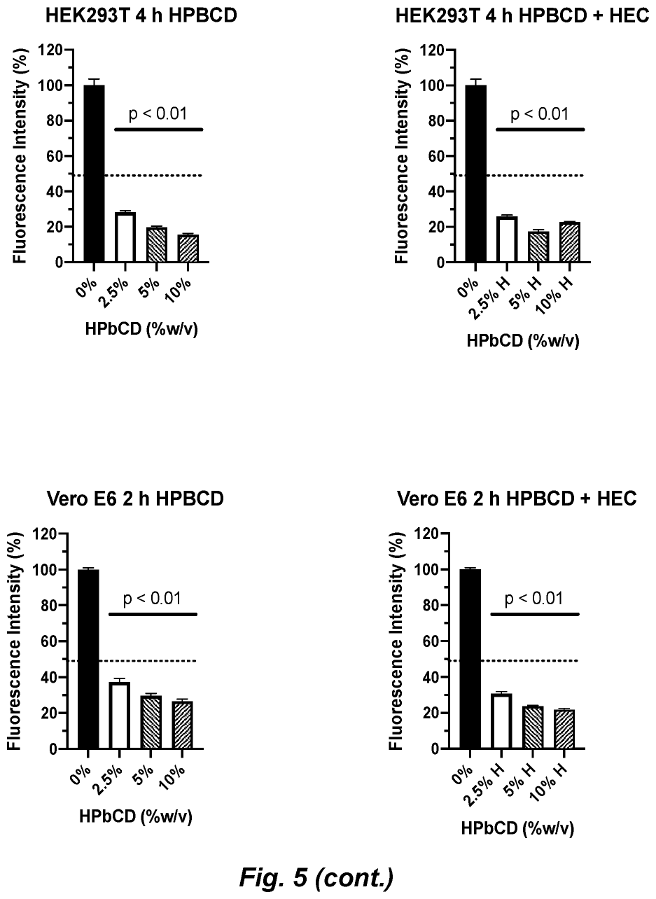

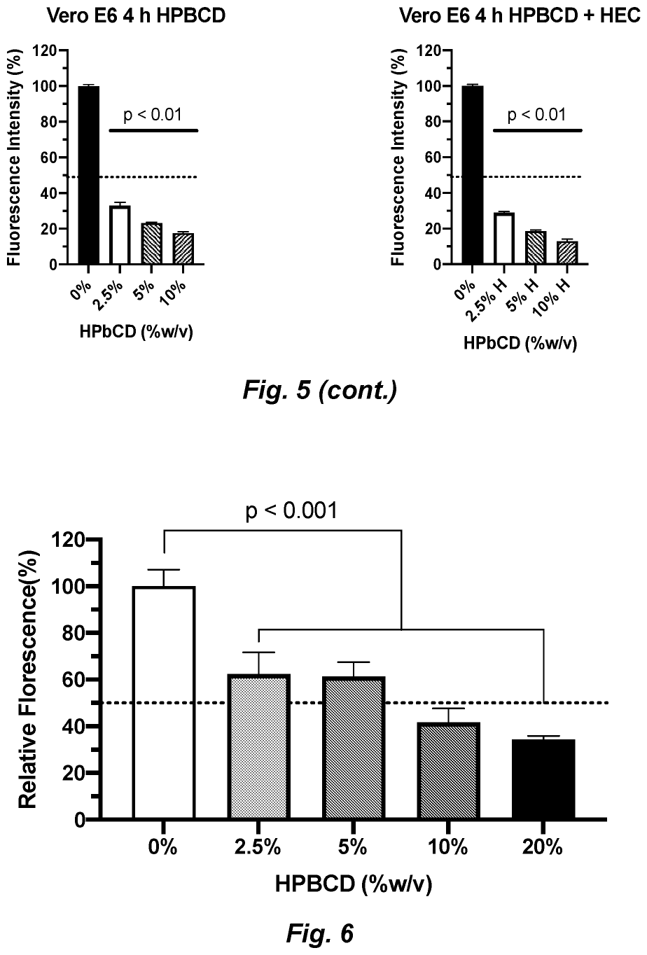

Studies test efficacy barrier to prevent chemical dye penetration. We used a resurazine dye that can penetrate across cellular membranes where it is oxidized to form a purple color and is quantifiable using fluorescence or visible light. Chemical exposure used Alamar Blue which was incubated for 4 hours. The ability of the barrier formulation to prevent chemical penetration was measured at 570 nm and confirmed using fluorescence intensity (560/590 nm) readings. A concentration dependent response to HPBCD was observed over four hours of incubation. The chemical dye penetration was reduced with increasing percent beta cyclodextrin across all timepoints (FIG. 5). The presence of HEC resulted in an improvement of the barrier to resist chemical penetration (Table 1). In addition to increased efficacy, 1.5% HEC addition has been shown to be more tolerable and have a protective effect upon the cells in vitro.

HPBCD formulations with and without HEC were tested across multiple cell lines (HEK293T, Vero-E6 ACE2). After two and four hours of incubation with Alamar blue dye, fluorescence readout show a significant concentration-dependent decrease in dye penetration. Compared to the media control, there is a 62-89% decrease in fluorescence output across cell lines and timepoints. In all cell lines, greater than 50% reduction in fluorescence intensity is observed using 2.5% HPBCD in both formulations with and without 1.5% HEC. The reduction was comparable between HEK293T and Vero-E6 ACE2 (FIG. 5). This affirms the improved efficacy of the barrier in preventing penetration in a respiratory cell line compared to a non-respiratory cell line. The utility of this finding extends the efficacy of the barrier from microbes even to chemical agents that may be toxic to the human cells.

TABLE 1
HPBCD Nanoscale Barrier reduces chemical penetration without
and with 1.5% HEC. Values displayed indicate percent reduction
compared to the media-treated negative control.

| | |
|---|---|
| | Percent Reduction Compared to No Barrier Treatment |
| Time (hr) | (Mean ± SD) |

A549

| | | | |
|---|---|---|---|
| HPBCD only | 2.5% HPBCD | 5% HPBCD | 10% HPBCD |
| 2 | 82.5 ± 1.4 | 88.5 ± 0.7 | 89.3 ± 0.6 |
| 4 | 66.9 ± 1.9 | 81.5 ± 0.7 | 82.9 ± 0.9 |
| Combination | 2.5% + 0.188% HEC | 5% + 0.375% HEC | 10% + 0.75% HEC |
| 2 | 86.0 ± 0.4 | 89.1 ± 3.6 | 84.2 ± 2.7 |
| 4 | 71.7 ± 0.4 | 81.8 ± 1.3 | 77.8 ± 0.9 |

HEK293T

| | | | |
|---|---|---|---|
| HPBCD only | 2.5% | 5% | 10% |
| 2 | 70.4 ± 1.7 | 77.5 ± 0.6 | 79.8 ± 0.6 |
| 4 | 71.7 ± 0.8 | 80.4 ± 0.8 | 84.3 ± 0.6 |
| Combination | 2.5% + 0.188% HEC | 5% + 0.375% HEC | 10% + 0.75% HEC |
| 2 | 75.2 ± 1.3 | 80.4 ± 2.4 | 71.2 ± 3.9 |
| 4 | 74.0 ± 0.7 | 82.5 ± 1.0 | 77.4 ± 0.4 |

Vero-E6 ACE2

| | | | |
|---|---|---|---|
| HPBCD only | 2.5% | 5% | 10% |
| 2 | 62.8 ± 2.0 | 70.2 ± 1.2 | 73.5 ± 1.2 |
| 4 | 67.1 ± 1.8 | 76.9 ± 0.6 | 82.5 ± 0.9 |
| Combination | 2.5% + 0.188% HEC | 5% + 0.375% HEC | 10% + 0.75% HEC |
| 2 | 69.2 ± 1.1 | 76.5 ± 0.6 | 78.4 ± 0.9 |
| 4 | 71.2 ± 0.7 | 81.4 ± 0.5 | 87.2 ± 1.3 |

### Efficacy of Barrier in Other Cyclodextrins

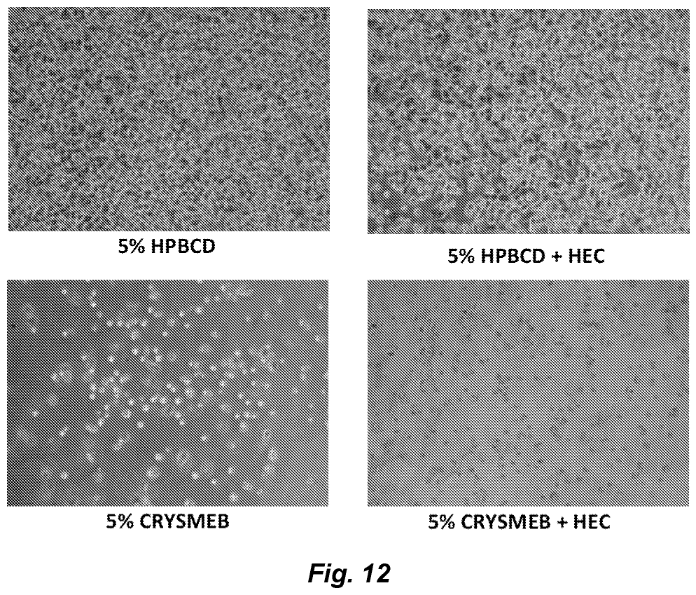

The ability to block chemical exposure was also conducted in the other cyclodextrins namely hydroxypropyl gamma cyclodextrin (HPGCD), sulfonylbutyl ethyl beta cyclodextrins (SBEBCD), and randomly methylated methyl beta cyclodextrins (CRYSMEB) and incubating over 4 hours. The ability of the barrier formulations to prevent chemical penetration was measured at an absorbance of 570 nm and confirmed using fluorescence intensity (560/590 nm) readings. Unlike HPBCD, when HPGCD and SBEBCD was used as nano formulation, these barriers enhanced chemical dye penetration in confluent A549 cells. At 2 h timepoint, SBEBCD showed a dose-dependent reduction in chemical dye penetration. Similarly, chemical dye penetration was reduced in a dose-dependent manner across all timepoints when CRYSMEB barrier was applied (FIG. 12). The addition of HEC improved the ability to prevent chemical penetration (Table 2). Although the data is unexpected, it allows for further probe into the effect of these cyclodextrin derivatives on viral particles which may be bigger and more hydrophobic.

TABLE 2
The effect of HPGCD, CRYSMEB and SBEBCD Nanoscale Barrier Without
and HEC on chemical penetration. Values displayed indicate percent
reduction compared to the media-treated control. Negative values
indicate an increase in fluorescence intensity (excited at 488
nm and emitted at 510 nm) as compared to untreated control.

| | |
|---|---|
| | Percent Reduction Compared to No Barrier Treatment |
| Time (hr) | (Mean ± SD) |

HPGCD

| | | | |
|---|---|---|---|
| HPGCD only | 2.5% | 5% | 10% |
| 2 | −89.9 ± 3.8* | −46.7 ± 2.4* | −13.1 ± 1.4* |
| 4 | −244.5 ± 3.0* | −210.66 ± 3.0 | −97.0 ± 2.1* |
| HPGCD + HEC | 2.5% + 0.188% HEC | 5% + 0.375% HEC | 10% + 0.75% HEC |
| 2 | −92.5 ± 25.5* | −17.6 ± 43.6* | 48.4 ± 0.6 |
| 4 | 48.6 ± 34.7 | 66.6 ± 46.3 | 63.4 ± 14.6 |

SBEBCD

| | | | |
|---|---|---|---|
| SBEBCD only | 2.5% | 5% | 10% |
| 2 | 10.7 ± 2.57 | 39.5 ± 1.2 | 51.3 ± 2.4 |
| 4 | −244.5 ± 3.0* | −218.2 ± 13.2* | −108.2 ± 19.5* |
| SBEBCD + HEC | 2.5% + 0.188% HEC | 5% + 0.375% HEC | 10% + 0.75% HEC |
| 2 | 26.8 ± 11.2 | 47.51 ± 6.3 | 60.1 ± 1.5 |
| 4 | −252.4 ± 34.7 | −185.0 ± 46.3 | −39.2 ± 14.5 |

CRYSMEB

| | | | |
|---|---|---|---|
| CRYSMEB only | 2.5% | 5% | 10% |
| 2 | 36.0 ± 2.3 | 47.9 ± 0.8 | 58.5 ± 1.4 |
| 4 | 35.3 ± 1.9 | 52.3 ± 2.6 | 67.6 ± 2.3 |
| CRYSMEB + HEC | 2.5% + 0.188% HEC | 5% + 0.375% HEC | 10% + 0.75% HEC |
| 2 | 34.4 ± 8.7 | 54.7 ± 3.8 | 64.9 ± 1.0 |
| 4 | 19.3 ± 7.2 | 40.7 ± 2.6 | 47.3 ± 1.3 |

### Ability of Nanoscale Barrier to Prevent Viral Infection

To simulate viral infection, we used lentivirus that has green fluorescent protein (GFP) incorporated into its gene. Lentivirus are designed to be highly efficient to transfect epithelial cells such as HEK293T. The effect of HPBCD barrier to present viral infection was evaluated using a concentration escalation study, where HPBCD ranged from 0 to 25% (w/v). In this study, we used a pLV[Exp]-Puro-CMV>EGFP lentivirus to infect HEK293T. We found that a multiplicity of infectivity (MOI) was able to effectively infected HEK293T. The ability of HPBCD barriers were then tested using this system, where after 6 hours the impact of HPBCD is summarized in FIG. 5. Despite using a MOI of 5, the barriers reduced lentivirus infectivity at 10% and 20% HPBCD (p≤0.01).

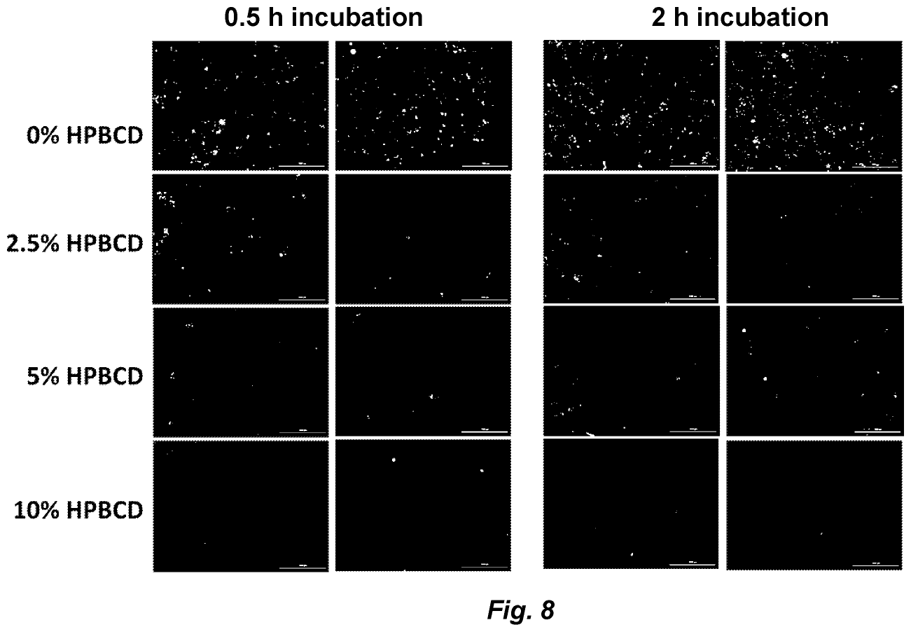

Human muco-ciliary deposition and clearance of exogenous substances occurs in 30 minutes as determined through real time methodology (Thrall et al., Inhalation Toxicology, 2003. 15(6): p. 523-538). Subsequently the EGFP lentivirus was incubated in the presence and absence of the HPBCD barrier for reduced time periods (0.5 h and 2 h) to reflect applicability of a HPBCD nasal spray. After the incubation period, barriers were removed, and cells were washed with 1×PBS before replacement with complete cell culture medium. 24 hours post-incubation at 5% CO₂ and 37 degrees C., fluorescence imaging was conducted using the Biotek Cytation 5 imaging device in which 4×GFP images were taken. In both the 0.5 h and 2 h incubation, a similar concentration dependent decrease in infectivity in HEK cells was observed (p<0.05 and p<0.01 respectively; FIG. 7 & FIG. 8). After 30 minutes of incubation with the virus, there is 50% reduction of viral infectivity as measured by fluorescence signal using 5% HPBCD.

### Addition of Zinc and Ascorbic Acid to HPBCD Barrier

Zinc and ascorbic acid have been associated with antiviral activity, where its supplementation has been highly recommended. We investigate the impact of adding ascorbic acid and/or zinc to determine whether the addition will enhance antiviral activity (Read et al., Advances in Nutrition, 2019. 10(4): p. 696-710; Kumar et al., Medical hypotheses, 2020. 144: p. 109848-109848). Zinc has been suggested to have immunomodulatory effect that include the ability to stabilizing the host cell membrane to reduce viral entry. As such, zinc was added the beta cyclodextrin formulation (3 mg/mL in 10% HPBCD) to determine its effect upon viral penetration when applied as a physical barrier. Ascorbic acid is another supplement that has demonstrated potential antiviral activity clinically and was subsequently added to the formulation as well (9 mg/mL in 10% HPBCD). In addition, lower pH (3.5-6.5) has been shown to inhibit viral infectivity and survival and thus presents as another variable for antiviral activity. The ascorbic acid was titrated to obtain the right concentration that will be safe for human mucocutaneous membranes (with guidance from recommended pH of nasal sprays being 4.5 to 6.5). The results in FIG. 8 indicated the resulting pH after preparing the nano formulation barrier in media for in vitro testing. The optimal HPBCD formulation that achieve pH within the range of 4.5 to 6.5 was tested for its efficacy to prevent viral infectivity, hoping to achieve synergy from the HPBCD, zinc, ascorbic acid and the pH. The ratio of HPBCD to zinc to ascorbic acid in the 10% HPBCD formulation was 100:3:9. These concentrated formulations were diluted with water to achieve lower concentrations.

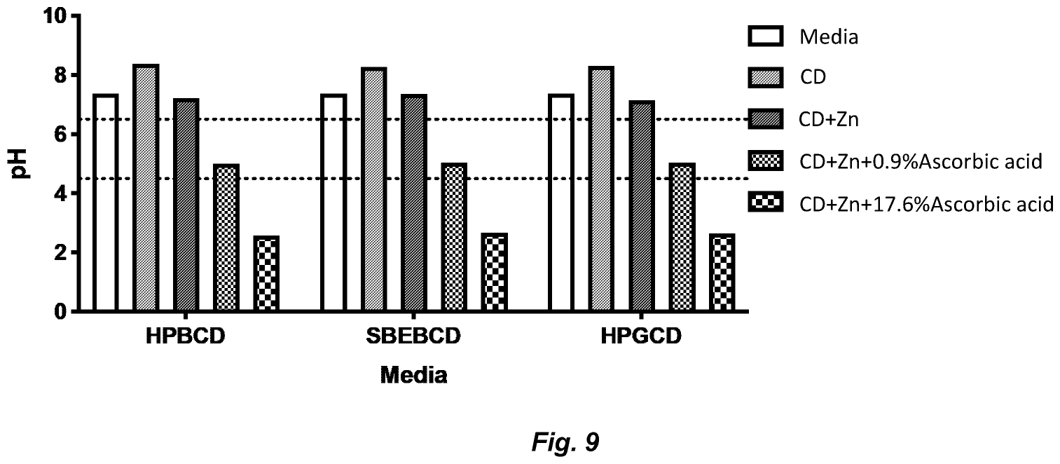

The results in FIG. 9, show that the addition of zinc and ascorbic acid resulted in an increase in relative fluorescence intensity associated with viral infectivity when admixed with 2.5% HPBCD at the predefined ratio of 100:3:9. Using higher concentrations of HPBCD, a slight decrease in fluorescence was observed as compared to control using 5% and 10% beta cyclodextrin (FIG. 8). These differences, however, were not statistically significant. Compared to the formulation without these additions, zinc and ascorbic acid have a negative effect upon reducing viral infectivity. This could potentially be due to interference with cholesterol sequestration by interfering with the binding sites of HPBCD pockets. However, higher concentrations of HPBCD tend to have more free binding sites of HPBCD pockets to effect inactivation on the viral particles. Incubation of the formulation with zinc and ascorbic acid additives resulted in significant reduction in viral transmissivity. However, the 2.5% formulation lacked efficacy when the viral cells were cultured over 24 hours post incubation with the barrier. Further studies will be conducted to validate these findings and the mechanisms.

### Safety of Cyclodextrin Based Barriers

During protection of mucocutaneous linings, barriers will be exposed repeatedly due to frequent dosing. A critical criterion for selecting the right cyclodextrin for a nanoformulation barrier usable in this application is the safety of the cyclodextrin type. A549 cells were seeded at 4×10⁴ cells per well and grown to confluence. Cyclodextrin-based formation candidates were tested at 5% concentrations and incubated for 1 h prior to the addition. The barrier was then replaced with media for evaluation. Pictures were taken for at least five field views at 10× magnification.

### Pharmaceutical Formulations

The compounds described herein can be used to prepare therapeutic pharmaceutical compositions, for example, by combining the compounds with a pharmaceutically acceptable diluent, excipient, or carrier. The compounds may be added to a carrier in the form of a salt or solvate. For example, in cases where compounds are sufficiently basic or acidic to form stable nontoxic acid or base salts, administration of the compounds as salts may be appropriate. Examples of pharmaceutically acceptable salts are organic acid addition salts formed with acids that form a physiologically acceptable anion, for example, tosylate, methanesulfonate, acetate, citrate, malonate, tartrate, succinate, benzoate, ascorbate, α-ketoglutarate, and β-glycerophosphate. Suitable inorganic salts may also be formed, including hydrochloride, halide, sulfate, nitrate, bicarbonate, and carbonate salts.

Pharmaceutically acceptable salts may be obtained using standard procedures well known in the art, for example by reacting a sufficiently basic compound such as an amine with a suitable acid to provide a physiologically acceptable ionic compound. Alkali metal (for example, sodium, potassium or lithium) or alkaline earth metal (for example, calcium) salts of carboxylic acids can also be prepared by analogous methods.

The compounds of the formulas described herein can be formulated as pharmaceutical compositions and administered to a mammalian host, such as a human patient, in a variety of forms. The forms can be specifically adapted to a chosen route of administration, e.g., oral or parenteral administration, by intravenous, intramuscular, topical or subcutaneous routes.

The compounds described herein may be systemically administered in combination with a pharmaceutically acceptable vehicle, such as an inert diluent or an assimilable edible carrier. For oral administration, compounds can be enclosed in hard- or soft-shell gelatin capsules, compressed into tablets, or incorporated directly into the food of a patient's diet. Compounds may also be combined with one or more excipients and used in the form of ingestible tablets, buccal tablets, troches, capsules, elixirs, suspensions, syrups, wafers, and the like. Such compositions and preparations typically contain at least 0.1% of active compound. The percentage of the compositions and preparations can vary and may conveniently be from about 0.5% to about 60%, about 1% to about 25%, or about 2% to about 10%, of the weight of a given unit dosage form. The amount of active compound in such therapeutically useful compositions can be such that an effective dosage level can be obtained.

The tablets, troches, suppositories, pills, capsules, and the like may also contain one or more of the following: binders such as gum tragacanth, acacia, corn starch or gelatin; excipients such as dicalcium phosphate; a disintegrating agent such as corn starch, potato starch, alginic acid and the like; and a lubricant such as magnesium stearate. A sweetening agent such as sucrose, fructose, lactose or aspartame; or a flavoring agent such as peppermint, oil of wintergreen, or cherry flavoring, may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propyl parabens as preservatives, a dye and flavoring such as cherry or orange flavor. Any material used in preparing any unit dosage form should be pharmaceutically acceptable and substantially non-toxic in the amounts employed. In addition, the active compound may be incorporated into sustained-release preparations and devices.

The active compound may be administered intravenously or intraperitoneally by infusion or injection. Solutions of the active compound or its salts can be prepared in water, optionally mixed with a nontoxic surfactant. Dispersions can be prepared in glycerol, liquid polyethylene glycols, triacetin, or mixtures thereof, or in a pharmaceutically acceptable oil. Under ordinary conditions of storage and use, preparations may contain a preservative to prevent the growth of microorganisms.

Pharmaceutical dosage forms suitable for injection or infusion can include sterile aqueous solutions, dispersions, or sterile powders comprising the active ingredient adapted for the extemporaneous preparation of sterile injectable or infusible solutions or dispersions, optionally encapsulated in liposomes. The ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions, or by the use of surfactants. The prevention of the action of microorganisms can be brought about by various antibacterial and/or antifungal agents, for example, parabens, chlorobutanol, phenol, sorbic acid, thimerosal, and the like. In many cases, it will be preferable to include isotonic agents, for example, sugars, buffers, or sodium chloride. Prolonged absorption of the injectable compositions can be brought about by agents delaying absorption, for example, aluminum monostearate and/or gelatin.

Sterile injectable solutions can be prepared by incorporating the active compound in the required amount in the appropriate solvent with various other ingredients enumerated above, as required, optionally followed by filter sterilization. In the case of sterile powders for the preparation of sterile injectable solutions, methods of preparation can include vacuum drying and freeze-drying techniques, which yield a powder of the active ingredient plus any additional desired ingredient present in the solution.

For topical administration, compounds may be applied in pure form, e.g., when they are liquids. However, it will generally be desirable to administer the active agent to the skin as a composition or formulation, for example, in combination with a dermatologically acceptable carrier, which may be a solid, a liquid, a gel, or the like.

Useful solid carriers include finely divided solids such as talc, clay, microcrystalline cellulose, silica, alumina, and the like. Useful liquid carriers include water, dimethyl sulfoxide (DMSO), alcohols, glycols, or water-alcohol/glycol blends, in which a compound can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using a pump-type or aerosol sprayer.

Thickeners such as synthetic polymers, fatty acids, fatty acid salts and esters, fatty alcohols, modified celluloses, or modified mineral materials can also be employed with liquid carriers to form spreadable pastes, gels, ointments, soaps, and the like, for application directly to the skin of the user.

Examples of dermatological compositions for delivering active agents to the skin are known to the art; for example, see U.S. Pat. No. 4,992,478 (Geria), U.S. Pat. No. 4,820,508 (Wortzman), U.S. Pat. No. 4,608,392 (Jacquet et al.), and U.S. Pat. No. 4,559,157 (Smith et al.). Such dermatological compositions can be used in combinations with the compounds described herein where an ingredient of such compositions can optionally be replaced by a compound described herein, or a compound described herein can be added to the composition.

Inhaled, intranasal, washes, eyedrops, or ocular washes may be delivered by specialized devices using the guidance of simulation. The formulation can be a solution or powdered stored within the device and is extracted from the place of storage upon actuation, of the device, whereupon the powder or solution that can be expelled from the device in the form of a plume of powder or aqueous spray which is to be inhaled, intranasal delivery, or ocular administration as a drop or wash by the subject. Some DPIs have a powder reservoir and doses of the powder are measured out within the device. These reservoir devices may be less favored where the treatment is likely to be one or a small number of doses in an isolated treatment.

Useful dosages of the compositions described herein can be determined by comparing their in vitro activity, and in vivo activity in animal models. Methods for the extrapolation of effective dosages in mice, and other animals, to humans are known to the art; for example, see U.S. Pat. No. 4,938,949 (Borch et al.). The amount of a compound, or an active salt or derivative thereof, required for use in treatment will vary not only with the particular compound or salt selected but also with the route of administration, the nature of the condition being treated, and the age and condition of the patient, and will be ultimately at the discretion of an attendant physician or clinician.

In general, however, a suitable dose will be in the range of from about 0.5 to about 100 mg/kg, e.g., from about 10 to about 75 mg/kg of body weight per day, such as 3 to about 50 mg per kilogram body weight of the recipient per day, preferably in the range of 6 to 90 mg/kg/day, most preferably in the range of 15 to 60 mg/kg/day.

The compound is conveniently formulated in unit dosage form; for example, containing 5 to 1000 mg, conveniently 10 to 750 mg, most conveniently, 50 to 500 mg of active ingredient per unit dosage form. In one embodiment, the invention provides a composition comprising a compound or composition described herein formulated in such a unit dosage form.

The compound can be conveniently administered in a unit dosage form, for example, containing 5 to 1000 mg/m², conveniently 10 to 750 mg/m², most conveniently, 50 to 500 mg/m² of active ingredient per unit dosage form. The desired dose may conveniently be presented in a single dose or as divided doses administered at appropriate intervals, for example, as two, three, four or more sub-doses per day. The sub-dose itself may be further divided, e.g., into a number of discrete loosely spaced administrations.

The desired dose may conveniently be presented in a single dose or as divided doses administered at appropriate intervals, for example, as two, three, four or more sub-doses per day. The sub-dose itself may be further divided, e.g., into a number of discrete loosely spaced administrations, such as multiple inhalations from an insufflator or by application of a plurality of drops into the eye.

### EXAMPLES

### Example 1. Materials and Methods

### Modeling of Nasopharyngeal Cavity for Optimized Barrier Properties

Recent studies have indicated that COVID-19 virus infectivity and replication efficiency vary markedly from proximal airway to alveolar respiratory regions, with nasal epithelial cells being the most prone to infectivity. Consequently, modeling of the proximal airway was necessary to guide the determination of the optimal parameters for an aerosolized HPBCD nanoscale barrier in the nasal cavity. To do so, we first selected a 3D model of human nasal cavity using average geometry and dimensions to generate an average nasal model. We then developed a computational fluid dynamics (CFD) model using the COMSOL platform applied to this 3D mesh and validated our results using various results reported in the literature. Subsequently, we established a methodology to quantify deposition on the mucocutaneous walls lining the nasal cavity. This is to simulate parameters for viral particle attachment onto host cell receptors. The deposition of the aerosolized particles onto 3D nasal cavity are represented by over 125,000 small triangles; when a particle lands on a triangle, this subregion of the surface is considered either contaminated (in the case of a viral particle) or protected (in the case of a sprayed HPBCD particle). This quantification method is further applied to characterize the deposition profile obtained using particles of different sizes to identify hot zones of deposition depending on particle sizes. Notably, the same quantification method was also instrumental in evaluating the effects of multiple spraying parameter values on deposition to help establish an optimal product nasal spray profile to ensure the best deposition profile that will prevent viral infection. Our exploration so far has allowed us to characterize deposition while varying sprayed particle size, particle size distribution function, sprayed volume, spray velocity, spray cone angle, spray head insertion depth and concurrent inhalation speed; this computational exploration resulted in a drastic reduction of the parameter space to be explored to yield best protection.

### Chemicals and Materials

2-hydroxypropyl beta cyclodextrin (parenteral grade) purchased from Roquette America Inc (Keokuk, Iowa) and the hydroxyethyl cellulose (HEC; Natrosol 250 HHX Pharma) was acquired from Ashland Global Specialty Chemicals Inc. (Ashland, Ky.). Phosphate buffered saline (PBS, Corning Life Sciences, Tewksbury, Mass.), heat inactivated fetal bovine serum (FBS), phenol-free DMEM (4.5 g/L glucose) media (Corning Life Sciences, Tewksbury, Mass.), Resazurin sodium salt (Alamar blue, Sigma), 0.25% Trypsin-EDTA (Sigma) were purchased from the following their respective vendors. The lentivirus, pLV[Exp]-Puro-CMV>EGFP lentivirus and 5 mg/mL polybrene purchased from Vector Builder. Luciferase assay reagents (substrate, buffer, 5× cell culture lysis buffer) purchased from Promega.

### Barrier Formulation

The nano formulation barrier candidates were prepared and classified according to the number of iterations to facilitate formulation optimization. The first-iteration formulation consisted of a concentration ranging study from 0% to 20% HPBCD. This nano formulation has the low viscosity consistency to facilitate its utilization as potential candidates for intranasal and topical delivery.

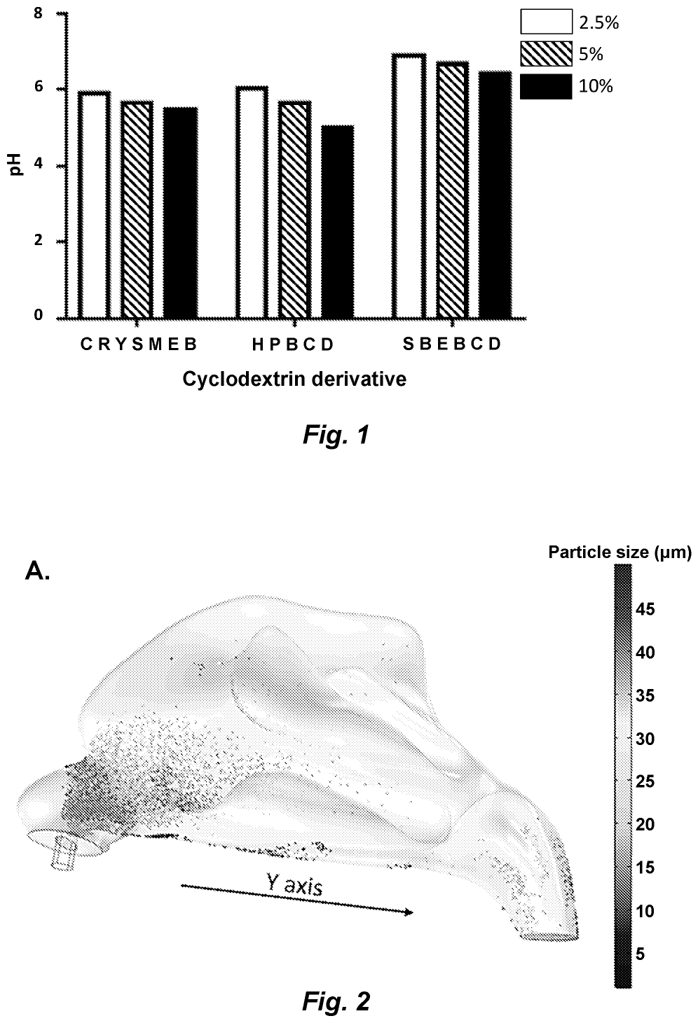

The selected cyclodextrins were formulated into a barrier in distilled water to evaluate their pH ranges thereby guiding their safe use in mammalian cells (FIG. 1). For in vitro testing, these nano formulation barriers were prepared in cell media to ensure the continuous supply of nutrients to the cells (Table 3). The barrier was prepared by mixing 10 g of HPBCD into about 30 mL of cell media in a 50 mL falcon tube. The mix was vortex until a homogenous solution was achieved. Then, a sufficient volume of cell media was added to the solution until the 50 mL mark. The solution was mixed for consistency and filtered through a 0.22 μm filter in a biosafety cabinet to achieve a sterile solution of 20% HPBCD barrier. Lower concentrations of 10%, 5%, and 2.5% HPBCD were achieved by diluting the 20% HPBCD with sterile cell media.

TABLE 3
Composition of the first iteration of the nano
formulation barrier prepared in phenol-free DMEM
to facilitate in vitro efficacy evaluations.

| Component | HPBCD | HPGCD | SBEBCD |
|---|---|---|---|
| Cyclodextrin (mg) | 10 | 10 | 10 |
| QSAD volume (mL) | 100 | 100 | 100 |

The second iteration of the nano formulation added excipients capable of increasing formulation viscosity. In this context, hydroxyethyl cellulose (HEC) was incorporated at a ratio consistent with HPBCD to enhance barrier retention at the targeted mucocutaneous surface (Table 4). The second iteration of nano formulation barriers were prepared by weighing the required amount of HEC into amount of required volume of water to make a 20% (v/v). A uniform dispersion of HEC in water was then heated at 100° C. for ten minutes. The mix was allowed to cool and the HPBCD content was added to solution and filtered through 0.22 μm filter to make the remaining 80% of the total formulation volume. The sterile HPBCD component in media was then incorporated into the sterilized HEC component to make the final sterile nano formulation for in vitro use.

TABLE 4
Composition of the second iteration of the HPBCD nano formulation
barrier prepared cell comparable medium with thickening
agent to facilitate in vitro efficacy evaluations.

| | 10% | 5% | 2.5% |
|---|---|---|---|
| Component | Formulation | Formulation | Formulation |
| HPBCD (g) | 10 | 5 | 2.5 |
| HEC (g) | 0.75 | 0.375 | 0.188 |
| QSAD volume (mL) | 100 | 100 | 100 |

### Effect of HPBCD on Chemical Penetration

To determine the ability of the candidate nanoscale barrier to prevent chemical penetration, epithelial cells (e.g., HEK293T, A549, Vero-E6 ACE2) were seeded at 1×10⁴ cells per well and incubated in DMEM till confluence. Cyclodextrin-based formation candidates were tested at various concentrations from 0 to 20% was added at varying concentrations (% w/v) and incubated for 0.5 h prior to the addition of the Alamar Blue. Alamar Blue is able to penetrate the cell, where viable cells are able to reduce the resazurin-based solution to quantitatively measure level of penetration.

### Effect of HPBCD on Lentivirus Infection

To test the ability to prevent viral Epithelial cells (e.g., HEK293T, A549, Vero-E6 ACE2) were used a models chemical and viral infections. Cells were seeded onto 96-well plates at a density of 10⁴ cells per well and incubated in DMEM (phenol red free) with 10% FBS and 1% non-essential ammino acids (NEAA). Cells were allowed to attach and grown to confluence to allow for cell polarization prior to experimentation.

Cyclodextrin was added at varying concentrations (% w/v) and incubated for 0.5 h prior to the addition of the virus. Polybrene added at a final concentration of 5 ug/mL to promote viral uptake. Cells were then infected with pLV[Exp]-Puro-CMV>EGFP lentivirus and incubated at 37° C., 5% CO₂ for 0.5, 2, or 24 h depending upon the experimental setup. After the incubation time, the beta cyclodextrin solution was removed and cells washed with 1×PBS twice prior to the addition of complete growth medium (DMEM). GFP lentivirus was imaged using the Cytation 5 (Biotek) and relative fluorescence quantified using the Synergy H1 microplate reader (Biotek).

### Infection (rVSV-SARS-CoV-2)

Vero-E6 ACE2 cells washed with 1×PBS, trypsinized, and seeded in treated 96 well plates at a density of 10⁴ and allowed to grow to confluence to allow for cell polarization prior to experimentation. Cell media is DMEM (phenol red free) with the addition of 10% FBS (no antibiotics). Cells were washed twice with 1×PBS (warmed to 37° C.) before adding beta cyclodextrin at varying concentrations (% w/v). Cells were incubated for 0.5 h prior to the addition of polybrene (5 ug/mL final concentration) and rVSV-SARS-CoV-2-Luc in serial dilution. Plate was incubated for 24/48 h and washed repeatedly with 1×PBS to remove the barrier formulation and replaced with complete growth medium.

### Preparation of Mammalian Cell Lysate (Luciferase Assay)

48 hours after incubation, growth medium was removed from cultured cells. Cells rinsed in 1×PBS and as much of the final wash was removed as possible. A minimal volume of 1× cell culture lysis buffer was added to each culture vessel and 50 uL of luciferase assay reagent (Promega) was added to the cell lysate and transferred to a white 96 well plate for the measurement of light production. Relative luminescence readings were obtained using the Synergy H1 microplate reader (Biotek).

### Example 2. Wearable Engineered Coating for the Prevention of Ophthalmic, Oral, and Nasopharyngeal Infections

Mucocutaneous membranes found in the eyes, nasal, respiratory, and gastrointestinal passages are susceptible to bacterial and viral infections. Coronaviruses have exploited these semi-permeable membranes to cause acute and severe infections. The severe acute respiratory syndrome coronavirus 2 (SARS-CoV-2) has affected over 13 million individuals in the U.S. leading to a 2% mortality rate. Host cell entry of the novel coronavirus is mediated by binding of the SARS-CoV-2 spike protein to host cell surface receptors including human angiotensin converting enzyme 2 (hACE2). In situ RNA mapping has revealed ACE2 expression to be highest in the nose and lower in the lung, pointing to nasal susceptibility as a predominant route for infection. Furthermore, the oral and ocular orifices serve as additional major ports for SARS-CoV-2. Current preventative measures for viral infection include handwashing, the use of personal protective equipment (PPE), and self-quarantine.

Applicants disclose a wearable engineered biocompatible coating capable of providing a barrier to the mucosal membranes to prevent viral and microbial attachment and entrapment. The system is based on hydroxypropyl beta cyclodextrin (HPBCD)-based technology, where its safety profile has been FDA-approved.

In silico 2D and 3D models have been established to simulate particle interactions with the human nasopharyngeal system. These models can simulate the effect of particle size, distribution, viscosity, and spray angle on barrier deposition to support the development of HPBCD-based barrier formulations. This model can establish the proportion of viral inoculum reduction in relation to concentration and time of exposure of the nanoformulation.

To verify these predictions, in vitro pseudotyped viral systems expressing the SARS-CoV-2 spike (S) protein are being developed to assess viral infectivity using luciferase/GFP as an indicator. Data presented herein utilizes chemical dyes and a GFP overexpressing lentivirus to assess barrier efficacy in reducing viral infectivity.

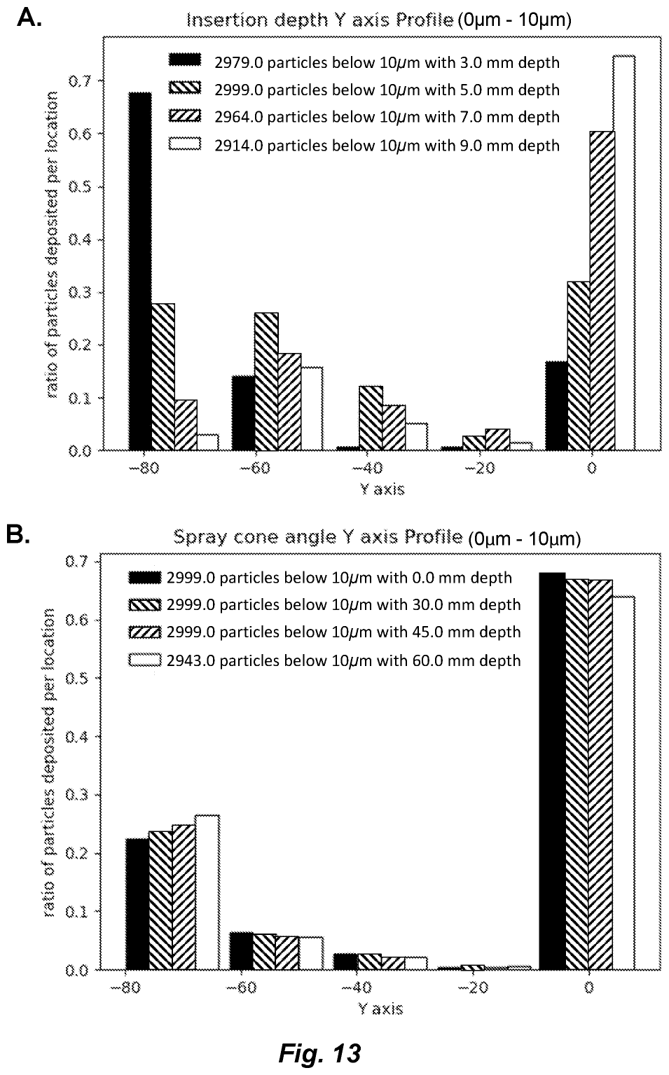

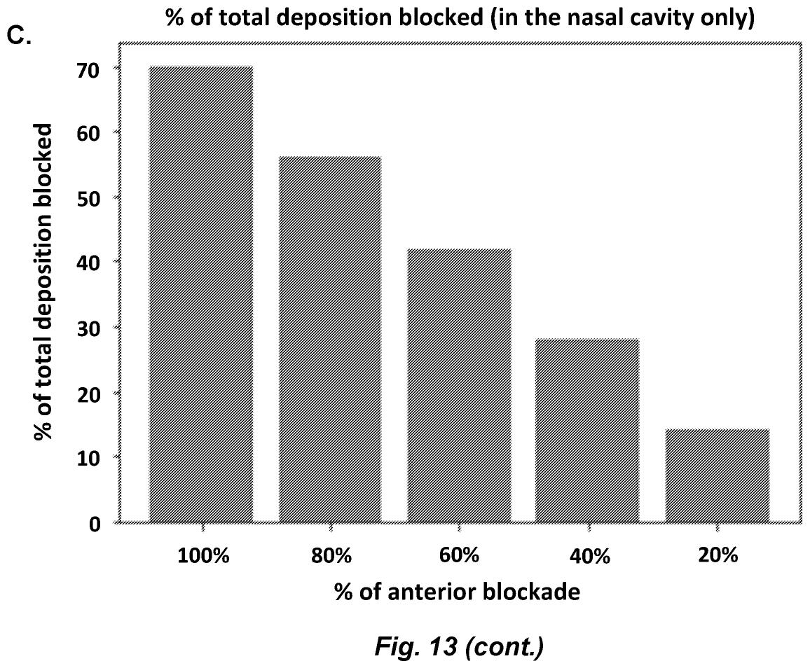

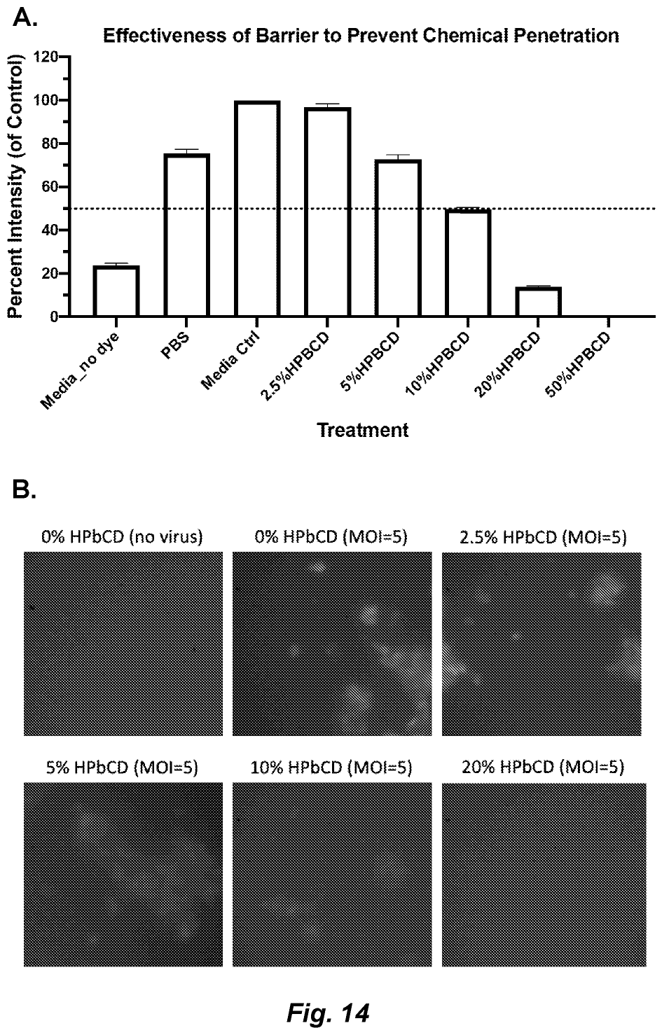

3D modeling of the human nasal passage reveal penetration depth is dependent upon the regionality of both viral and HPBCD deposition. Most viral particles and droplets that may reach far are likely to be below 10 microns so most of the modeling focused on this range. Higher proportion of particle deposition was observed with 3 mm insertion depth. However, neither the spray cone angle nor the spray velocity significantly changes the deposition profile of particles less than 10 micron in the nasal cavity. The anterior region needs about 70% block in the anterior region to reduce a 50% of the viral burden deposition. (FIG. 13A-12C) Furthermore, in vitro, the HPBCD barrier shows a concentration-dependent decrease in dye penetration as indicated by absorbance in HEK293T cells plated in a monolayer. The IC₅₀ of the dye penetration was found at 10% HPBCD. Utilizing GFP expressing lentiviruses, 5-20% HPBCD alone was able to reduce viral infectivity. No signs of lentivirus infection were observed using 20% HPBCD alone as a barrier. (FIG. 14A-13B).

### Example 3. Pharmaceutical Dosage Forms

The following formulations illustrate representative pharmaceutical dosage forms that may be used for the therapeutic or prophylactic administration of a composition or formulation of a formula described herein, a composition specifically disclosed herein, or a pharmaceutically acceptable salt or solvate thereof (hereinafter referred to as ‘Composition X’):

| (i) Aerosol | mg/can |
|---|---|
| ‘Composition X’ | 20 |
| Oleic acid | 10 |
| Trichloromonofluoromethane | 5,000 |
| Dichlorodifluoromethane | 10,000 |
| Dichlorotetrafluoroethane | 5,000 |
| (ii) Topical Gel 1 | wt. % |
| ‘Composition X’ | 5% |
| Carbomer 934 | 1.25% |
| Triethanolamine | q.s. |
| (pH adjustment to 5-7) | |
| Methyl paraben | 0.2% |
| Purified water | q.s. to 100 g |
| (iii) Topical Gel 2 | wt. % |
| ‘Composition X’ | 5% |
| Methylcellulose | 2% |
| Methyl paraben | 0.2% |
| Propyl paraben | 0.02% |
| Purified water | q.s. to 100 g |
| (iv) Topical Ointment | wt. % |
| ‘Composition X’ | 5% |
| Propylene glycol | 1% |
| Anhydrous ointment base | 40% |
| Polysorbate 80 | 2% |
| Methyl paraben | 0.2% |
| Purified water | q.s. to 100 g |
| (v) Topical Cream 1 | wt. % |
| ‘Composition X’ | 5% |
| White bees wax | 10% |
| Liquid paraffin | 30% |
| Benzyl alcohol | 5% |
| Purified water | q.s. to 100 g |
| (vi) Topical Cream 2 | wt. % |
| ‘Composition X’ | 5% |
| Stearic acid | 10% |
| Glyceryl monostearate | 3% |
| Polyoxyethylene stearyl ether | 3% |
| Sorbitol | 5% |
| Isopropyl palmitate | 2% |
| Methyl Paraban | 0.2% |
| Purified water | q.s. to 100 g |

These formulations may be prepared by conventional procedures well known in the pharmaceutical art. It will be appreciated that the above pharmaceutical compositions may be varied according to well-known pharmaceutical techniques to accommodate differing amounts and types of active ingredient ‘Composition X’. Aerosol formulation (vi) may be used in conjunction with a standard, metered dose aerosol dispenser. Additionally, the specific ingredients and proportions are for illustrative purposes. Ingredients may be exchanged for suitable equivalents and proportions may be varied, according to the desired properties of the dosage form of interest.

While specific embodiments have been described above with reference to the disclosed embodiments and examples, such embodiments are only illustrative and do not limit the scope of the invention. Changes and modifications can be made in accordance with ordinary skill in the art without departing from the invention in its broader aspects as defined in the following claims.

All publications, patents, and patent documents are incorporated by reference herein, as though individually incorporated by reference. No limitations inconsistent with this disclosure are to be understood therefrom. The invention has been described with reference to various specific and preferred embodiments and techniques. However, it should be understood that many variations and modifications may be made while remaining within the spirit and scope of the invention.

What is claimed is:

1. A formulation comprising:
a cyclodextrin derivative present in a concentration of about 2.5% to about 20% by weight of the formulation, wherein the cyclodextrin derivative is one or more selected from the group consisting of hydroxypropyl-beta-cyclodextrin, 2-hydroxypropyl-gamma-cyclodextrin, crystalline methylated-beta-cyclodextrin, and sulfobutyl-ether-beta-cyclodextrin; and
a pharmaceutically acceptable carrier.

2. The formulation of claim 1 wherein the cyclodextrin is hydroxypropyl-beta-cyclodextrin, 2-hydroxypropyl-gamma-cyclodextrin, or sulfobutyl-ether-beta-cyclodextrin.

3. The formulation of claim 2 wherein the cyclodextrin is hydroxypropyl-beta-cyclodextrin.

4. The formulation of claim 1 wherein the pharmaceutically acceptable carrier is a thickening agent, wherein a ratio of the cyclodextrin to the thickening agent is about 5:3 to about 15:1 by weight.

5. The formulation of claim 4 wherein the thickening agent is one or more of a cellulose polymer, a cellulose derivative, gelatin, polyvinylpyrrolidone, tragacanth, ethoxose, alginic acid, polyvinyl alcohol, polyacrylic acid, pectin, and a poloxamer.

6. The formulation of claim 5 wherein the thickening agent is the cellulose polymer or the cellulose derivative, wherein the cellulose polymer or the cellulose derivative is hydroxyethyl cellulose, methyl cellulose, carboxymethyl cellulose, or a combination thereof.

7. (canceled)

8. The formulation of claim 1 wherein a pH of the formulation is about 4.5 to about 7.5.

9. The formulation of claim 1 wherein the formulation forms a coating over a mucocutaneous lining, thereby preventing entry of a microbe or of a gaseous agent into the cell.

10. The formulation of claim 9 wherein the mucocutaneous lining is a surface of one or more of a nasal cavity, an oropharyngeal cavity, a gastrointestinal cavity, a bronchial cavity, a pulmonary cavity, an oral cavity, a rectal cavity, a vaginal cavity, or an ocular cavity.

11. The formulation of claim 9 wherein the microbe is a virus, a bacterium, or a fungus.

12. The formulation of claim 11 wherein the virus comprises one or more of human immunodeficiency virus (HIV), human metapneumovirus (HMPV), parainfluenza virus type 3 (HPIV3), Influenza virus A, Influenza virus B, Influenza virus C, Influenza virus D, coronavirus infectious bronchitis virus (IBV), herpes simplex virus 1 (HSV-1), herpes simplex II (HSV-2) varicella-zoster virus (VZV), Epstein Barre virus (EBV), cytomegalovirus (CMV), Kaposi's Sarcoma herpes virus (KSHS), hepatitis A virus (HAV), hepatitis C virus (HCV), hepatitis B virus (HBV), human papilloma virus (HPV), SARS-CoV1, Middle East Respiratory Syndrome (MERS) virus, and SARS-CoV-2 virus and variants.

13. The formulation of claim 12 wherein the virus is SARS-CoV-2 virus and variants thereof

14. The formulation of claim 1 wherein the formulation comprises 0.9% or less (w/v) of zinc or zinc containing compounds.

15. The formulation of claim 1 comprises an aerosol, inhalant, intranasal spray, eyedrop, gel, ointment, wash, lozenges, powder, tablets, capsules, pessaries, aqueous spray, or suppository.

16. A method of reducing a risk of a microbial infection in a subject comprising contacting cells in the subject susceptible to infection with an effective amount of the formulation of claim 1 to form a coating over a surface of the cells, the coating preventing the microbe from contacting the cells, thereby reducing the risk of the microbial infection in the subject.

17. The method of claim 16 wherein the formulation is an aerosol, inhalant, intranasal spray, or an aqueous spray, wherein the formulation comprises a use of a specialized device to contact the cells susceptible to infection with the formulation.

18. The method of claim 17 wherein the coating is formed on a mucocutaneous lining of a surface of one or more cavities selected from a nasal cavity, an oropharyngeal cavity, a gastrointestinal cavity, a bronchial cavity, a pulmonary cavity, an oral cavity, a rectal cavity, a vaginal cavity, or an ocular cavity.

19. The method of claim 16 wherein the causative agent of the microbial infection is a virus, a bacterium, a parasite, or a fungus.

20. The method of claim 19 wherein the virus comprises one or more of human immunodeficiency virus (HIV), human metapneumovirus (HMPV), parainfluenza virus type 3 (HPIV3), Influenza virus A, Influenza virus B, Influenza virus C, Influenza virus D, coronavirus infectious bronchitis virus (IBV), herpes simplex virus 1 (HSV-1), herpes simplex II (HSV-2) varicella-zoster virus (VZV), Epstein Barre virus (EBV), cytomegalovirus (CMV), Kaposi's Sarcoma herpes virus (KSHS), hepatitis A virus (HAV), hepatitis C virus (HCV), hepatitis B virus (HBV), human papilloma virus (HPV), SARS-CoV1, Middle East Respiratory Syndrome (MERS) virus, and SARS-CoV-2 virus and variants.

21. The method of claim 17 wherein the formulation is an aerosolized particulate delivered to the cells at a spray volume of about 50 μl to 160 μl, a spray velocity of about 4 m/s to 18 m/s, an insertion depth of about 10 mm into a cavity comprising the cells, and a spray cone angle of about π/4 to about π/2.

22. (canceled)

23. (canceled)
